(19) United States
(12) Patent Application Publication
(10) Pub. No.: US 2025/0049768 A1
(43) Pub. Date: Feb. 13, 2025

(54) METHODS OF TREATING ESTROGEN RECEPTOR-ASSOCIATED DISEASES

(71) Applicant: Olema Pharmaceuticals, Inc., San Francisco, CA (US)

(72) Inventors: Cyrus L. Harmon, Bolinas, CA (US); Peter J. Kushner, San Francisco, CA (US); David C. Myles, Berkeley, CA (US); Leslie Hodges Gallagher, Alameda, CA (US)

(21) Appl. No.: 18/576,880

(22) PCT Filed: Jul. 7, 2022
(86) PCT No.: PCT/US22/36351

§ 371 Date: Jan. 5, 2024

Related U.S. Application Data
(60) Provisional application No. 63/278,526, filed on Nov. 12, 2021.
(60) Provisional application No. 63/219,802, filed on Jul. 8, 2021.

(51) Int. Cl. A61K 31/437; A61K 31/517; A61K 31/519; A61P 35/00; A61P 35/04

(52) U.S. Cl. CPC A61K 31/437; A61K 31/517; A61K 31/519; A61P 35/00; A61P 35/04

(57) ABSTRACT

The present disclosure provides methods of treating a disease, disorder, or condition associated with ER+/HER2+ cancer using Compound 1, or a pharmaceutically acceptable salt thereof.

### RELATED APPLICATIONS

This application claims priority to U.S. Appl. No. 63/219,802, filed Jul. 8, 2021, and U.S. Appl. No. 63/278,526, filed Nov. 12, 2021, the entire contents of each of which are hereby incorporated by reference.

### BACKGROUND

The estrogen receptor (ER) plays important roles in various cancers, including breast cancers. A variety of treatments have been developed to target the estrogen receptor and/or its activities. Patients with estrogen receptor positive (ER+) and human epidermal growth factor 2-positive (HER2+) breast cancer are more likely to suffer from metastases to other organs, such as the brain, liver, and lung. Arciero, et al., Clin. Breast Cancer, 19(4):236-245 (2019). Cancer cells in the brain are particularly problematic. For hormone positive disease e.g., ER-positive breast cancer, the incidence of breast cancer brain metastases (BCBMs) is 14%, with a median overall survival after the development of brain metastases of 9-10 months. Brosnan, Ann Transl Med., 2016; 6(9):163. About 34% of patients with HER2+ breast cancer will develop central nervous system metastases, with a median survival of about 11-18 months. Id.

### SUMMARY

The present disclosure provides new insights regarding compounds and/or regimens useful for the treatment of estrogen receptor (ER)-associated diseases, disorders, and conditions (e.g., cancer cells) for subjects that exhibit both estrogen receptor positive (ER+) and human epidermal growth factor receptor positive (HER2+) disease, including disease that has metastasized to other organs, such as the brain. In some embodiments, the disease has metastasized to the brain, bones, lungs, or liver.

In some embodiments, the present disclosure encompasses an insight that certain therapies, e.g., complete estrogen receptor antagonists, are particularly useful for treating diseases, disorders, and conditions in subjects determined or otherwise suspected to exhibit both estrogen receptor positive and human epidermal growth factor positive associated disease (e.g., are ER+ and HER2+).

The benefit of complete estrogen receptor antagonists in treating estrogen receptor positive disease is described in PCT App. No. PCT/US21/21151, published as WO 2021/178846, which is incorporated herein by reference in its entirety. Previous reports, however, have not appreciated that certain complete estrogen receptor antagonists can be used in treatment of subjects who not only have ER+ disease but are also determined to be or are suspected to have HER2+ disease.

Among other things, the present disclosure provides methods of using such complete estrogen receptor antagonists (i.e., that cross the blood-brain barrier and/or are orally bioavailable), including specifically to detect, assess, and/or treat brain lesions (e.g., tumors such as metastases). In some embodiments, the present disclosure provides a method comprising a step of: administering to a subject determined to have an ER+ and HER2+ associated cancer a composition that comprises and/or delivers to the subject's brain (e.g., upon oral administration) a complete estrogen receptor antagonist, wherein the subject has been determined to have or is suspected of having brain metastases.

In some embodiments, the present disclosure teaches that Compound 1, or a pharmaceutically acceptable salt thereof, is a particularly useful complete estrogen receptor antagonist, with surprising activity in ER+ and HER2+ cell lines:

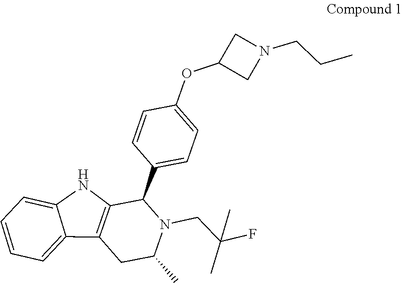 [SMILES: CCCN1CC(Oc2ccc([C@@H]3c4[nH]c5ccccc5c4C[C@@H](C)N3CC(C)(C)F)cc2)C1]

Further, the present disclosure encompasses an insight that Compound 1 can be advantageously administered in combination with a HER2 inhibitor. In some embodiments, a HER2 inhibitor is selected from tucatinib, pertuzumab, lapatinib, trastuzumab, ado-trastuzumab emtansine, trastuzumab deruxtecan, and neratinib.

### BRIEF DESCRIPTION OF THE DRAWING

FIG. 1A is a scatter plot measuring each animal's body weight at time 0. Throughout the Figures, “Cpd 1” refers to “Compound 1” as described herein.

FIG. 1B is a scatter plot measuring each animal's tumor volume at time 0.

FIG. 2 is a Kaplan-Meier plot showing the percentage of surviving mice in each Group over time. Tick marks indicate censored data reflecting that mice were enrolled on different days.

FIG. 3A is a scatter plot measuring mean tumor volume over time for the each Group.

FIG. 3B is a scatter plot measuring percent change in tumor volume over time for each Group.

FIG. 4A is a waterfall plot measuring the percent change in tumor volume for each Group.

FIG. 4B is a zoomed in waterfall plot of FIG. 4A, illustrating the decrease in tumor volume size for certain Groups.

FIG. 5 is a scatter plot measuring mean tumor volume over time for individual animals of each Group.

FIG. 6 is a scatter plot measuring percent change in tumor volume over time for individual animals of each Group.

FIG. 7 is a scatter plot measuring animal weight over time for each Group.

FIG. 8 is a scatter plot measuring percent change in animal weight over time for each Group.

FIG. 9 is a scatter plot measuring change in BT-474 xenograft mean volume over time.

FIG. 10 is a graph measuring concentration of therapy in plasma, tumor, and brain after 6 or 24 hours, demonstrating that Compound 1 exhibits brain penetrance.

FIG. 11A is a scatter plot measuring change in xenograft tumor volume over time for provided therapies. Kadcyla is otherwise known as ado-trastuzumab emtansine. Herceptin is otherwise known as trastuzumab.

FIG. 11B is a scatter plot measuring percent change in xenograft tumor volume over time.

FIG. 12A is a chart tracking HER2 immunoblot fluorescence relative to GAPDH in three cell lines, BT-474, ZR-75-1, and ZR-75-30 in a western blot of protein expression.

FIG. 12B is a chart tracking ERα fluorescence relative to GAPDH in three cell lines, BT-474, ZR-75-1, and ZR-75-30 in a western blot of protein expression.

FIG. 13 is a plot measuring cellular proliferation using fluorescence relative to control of a DNA-binding dye for varying doses of tucatinib in the BT-474 cell line after 7 days of compound treatment.

FIG. 14 is a plot measuring cellular proliferation using fluorescence relative to control of a DNA-binding dye for varying doses of tucatinib in the ZR-75-30 cell line after 7 days of compound treatment.

FIG. 15 is a plot measuring cellular proliferation using fluorescence relative to control of a DNA-binding dye for varying doses of tucatinib in the ZR-75-1 cell line after 7 days of compound treatment.

FIG. 16A is a bar graph illustrating ER-α protein quantification in BT-474, MDA-MB-361, and ZR-75-30 cell lines for certain compounds and combination.

FIG. 16B is a bar graph illustrating HER2 protein quantification in BT-474, MDA-MB-361, and ZR-75-30 cell lines for certain compounds and combination.

FIG. 16C is a scatter plot illustrating proliferation relative to vehicle in a BT-474 proliferation assay.

FIG. 16D is a scatter plot illustrating proliferation relative to vehicle in a MDA-MB-361 proliferation assay.

FIG. 16E is a scatter plot illustrating proliferation relative to vehicle in a ZR-75-30 proliferation assay.

FIG. 17A is a bar graph illustrating cell proliferation in a BT-474 cell line for certain compounds and combinations.

FIG. 17B is a bar graph illustrating cell proliferation in a MDA-MB-361 cell line for certain compounds and combinations.

FIG. 17C is a bar graph illustrating cell proliferation in a ZR-75-30 cell line for certain compounds and combinations.

FIG. 18A is a scatter plot measuring tumor volume in a BT-474 mammary fat pad xenograft for certain compounds and combinations.

FIG. 18B is an image of various tumors treated with vehicle, trastuzumab, or Compound 1 and trastuzumab.

FIG. 18C is a graph illustrating concentration of Compound 1, tucatinib, and trastuzumab in the plasma, tumor, and brain.

FIG. 18D is a plot measuring tumor volume for ER+/HER2+ tumors treated with various compounds and combinations.

FIG. 18E is a plot measuring tumor volume for ER+/HER2+ tumors treated with various compounds and combinations.

FIG. 19 is a plot measuring tumor volume in a BT-474 mammary fat pad xenograft for certain compounds and combinations.

FIG. 20 is a scatter plot illustrating proliferation relative to vehicle in a EFM192A proliferation assay.

FIG. 21 is a scatter plot measuring tumor volume in a CTG-2328 xenograft for certain compounds and combinations. The dotted line indicates mean tumor volume at Day 0.

FIG. 22 is a scatter plot measuring tumor volume in a CTG-2328 xenograft for certain compounds and combinations. The dotted line indicates mean tumor volume at Day 0.

FIG. 23 is a plot measuring tumor volume on Day 31 in a CTG-2328 xenograft for certain compounds and combinations.

FIG. 24 is a plot measuring tumor volume on Day 31 in a CTG-2328 xenograft for certain compounds and combinations.

### DETAILED DESCRIPTION OF CERTAIN EMBODIMENTS

The present disclosure provides methods of treating ER+/HER2+ associated diseases in a subject by administering a composition comprising a complete estrogen receptor antagonist, wherein the subject has been determined to have or is suspected of having a cancer that is both ER+ and HER2+. In some embodiments, the present application provides methods of treating a metastasis (e.g., a brain metastasis) related to ER+/HER2+ associated diseases in a subject by administering a composition comprising a complete estrogen receptor antagonist, wherein the subject has been determined to have or is suspected of having a cancer that is both ER+ and HER2+. In some embodiments, provided methods are for treating brain, bone, lung, or liver metastases. In some embodiments, the subject has been determined to have ER+/HER2+ disease and is determined to have or is suspected of having a metastasis, e.g., a brain metastasis, a bone metastasis, a lung metastasis, or a liver metastasis.

### Definitions

Administration: As used herein, the term “administration” typically refers to the administration of a composition to a subject or system, for example to achieve delivery of an agent that is, or is included in or otherwise delivered by, the composition.

Agent: As used herein, the term “agent” refers to an entity (e.g., for example, a lipid, metal, nucleic acid, polypeptide, polysaccharide, small molecule, etc., or complex, combination, mixture or system [e.g., cell, tissue, organism] thereof), or phenomenon (e.g., heat, electric current or field, magnetic force or field, etc.).

Antagonist: As used herein, the term “antagonist” may refer to an agent, or condition whose presence, level, degree, type, or form is associated with a decreased level or activity of a target. An antagonist may include an agent of any chemical class including, for example, small molecules, polypeptides, nucleic acids, carbohydrates, lipids, metals, and/or any other entity that shows the relevant inhibitory activity. In some embodiments, an antagonist may be a “direct antagonist” in that it binds directly to its target; in some embodiments, an antagonist may be an “indirect antagonist” in that it exerts its influence by means other than binding directly to its target; e.g., by interacting with a regulator of the target, so that the level or activity of the target is altered). In some embodiments, an “antagonist” may be referred to as an “inhibitor”.

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen or therapeutic regimen: Those skilled in the art will appreciate that the terms “dosing regimen” and “therapeutic regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses, each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Excipient: As used herein, the term “excipient” refers to a non-therapeutic agent that may be included in a pharmaceutical composition, for example, to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intra-arterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Patient or subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, non-human primates, and/or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, the active agent is present in unit dose amounts appropriate for administration in a therapeutic regimen to a relevant subject (e.g., in amounts that have been demonstrated to show a statistically significant probability of achieving a predetermined therapeutic effect when administered), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Pharmaceutically acceptable carrier: As used herein, the term “pharmaceutically acceptable carrier” means a pharmaceutically acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer.

In some embodiments, a small molecule does not include a polymeric moiety. In some embodiments, a small molecule is not and/or does not comprise a protein or polypeptide (e.g., is not an oligopeptide or peptide). In some embodiments, a small molecule is not and/or does not comprise a polynucleotide (e.g., is not an oligonucleotide). In some embodiments, a small molecule is not and/or does not comprise a polysaccharide; for example, in some embodiments, a small molecule is not a glycoprotein, proteoglycan, glycolipid, etc.). In some embodiments, a small molecule is not a lipid.

In some embodiments, a small molecule is a modulating agent (e.g., is an inhibiting agent or an activating agent). In some embodiments, a small molecule is biologically active. In some embodiments, a small molecule is detectable (e.g., comprises at least one detectable moiety). In some embodiments, a small molecule is a therapeutic agent.

Those of ordinary skill in the art, reading the present disclosure, will appreciate that certain small molecule compounds described herein may be provided and/or utilized in any of a variety of forms such as, for example, crystal forms (e.g., polymorphs, solvates, etc), salt forms, protected forms, pro-drug forms, ester forms, isomeric forms (e.g., optical and/or structural isomers), isotopic forms, etc.

Those of ordinary skill in the art will appreciate that certain small molecule compounds have structures that can exist in one or more stereoisomeric forms. In some embodiments, such a small molecule may be utilized in accordance with the present disclosure in the form of an individual enantiomer, diastereomer or geometric isomer, or may be in the form of a mixture of stereoisomers; in some embodiments, such a small molecule may be utilized in accordance with the present disclosure in a racemic mixture form.

Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms.

Those of skill in the art will appreciate that certain small molecule compounds have structures that permit isotopic substitution (e.g., ²H or ³H for H; ¹¹C, ¹³C or ¹⁴C for ¹²C; ¹³N or ¹⁵N for ¹⁴N; ¹⁷O or ¹⁸O for ¹⁶O; ³⁶Cl for ^{35/37}Cl; ¹⁸F for ¹⁹F; ¹³¹I for ¹²⁷I; etc). In some embodiments, such a small molecule may be utilized in accordance with the present disclosure in one or more isotopically modified forms, or mixtures thereof.

In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound); in some such embodiments, the salt form may be a pharmaceutically acceptable salt form.

In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound may be considered to be a different form of the compound than a racemic mixture of the compound; a particular salt of a small molecule compound may be considered to be a different form from another salt form of the compound; a preparation that contains only a form of the compound that contains one conformational isomer ((Z) or (E)) of a double bond may be considered to be a different form of the compound from one that contains the other conformational isomer ((E) or (Z)) of the double bond; a preparation in which one or more atoms is a different isotope than is present in a reference preparation may be considered to be a different form; etc.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Treat: As used herein, the terms “treat,” “treatment,” or “treating” refer to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example, for the purpose of decreasing the risk of developing pathology associated with the disease, disorder, and/or condition.

Therapeutically effective amount: As used herein, the term “therapeutically effective amount” refers to an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder and/or condition. In some embodiments, a therapeutically effective amount is administered in a single dose; in some embodiments, multiple unit doses are required to deliver a therapeutically effective amount.

It is understood by one skilled in the art that compounds referred to herein may be enriched at any or all atoms above naturally occurring isotopic ratios with one or more isotopes such as, but not limited to, deuterium (²H or D).

The compounds of the invention, or their pharmaceutically acceptable salts, may contain chiral centers, which, unless specified otherwise, may be either of the (R) or (S) configuration, or which may comprise a mixture thereof. Accordingly, the present application includes stereoisomers of the compounds described herein, where applicable, either individually or admixed in any proportions. Stereoisomers may include, but are not limited to, enantiomers, diastereomers, racemic mixtures, and combinations thereof. Such stereoisomers can be prepared and separated using conventional techniques, either by reacting enantiomeric starting materials, or by separating isomers of compounds of the present application.

### Estrogen Receptor and Human Epidermal Growth Factor 2 Associated Diseases and Disorders

### The Role of the Estrogen Receptor and Human Epidermal Growth Factor 2

The estrogen receptor (“ER”) is involved in a variety of biological processes, relating, for example, to development of the female reproductive system, maintenance of bone mass, protection of cardiovascular and/or central nervous system components, etc. (see, for example, Pearce & Jordan Crit. Rev. Onc/Hem 50:3, 2004; Heldring Phys. Rev. 87:905, 2007). The ER has been implicated in a variety of cancers. In many tumors that express the estrogen receptor (i.e., ER+ tumors), active ERα signaling has been demonstrated to drive cell proliferation (although ERβ signaling has been reported to be able to achieve tumor suppressor effects; see, for example, Nilsson & Gustafson Clin. Pharmacol. Ther. 89:44, 2011). Typically, tumors (e.g., breast tumors) with as few as 1% of cells staining positive for ER are classified as “ER+”. Therapies targeting the ER are standard of care for many patients with ER+ tumors (see, for example, Cardoso et al Annals Onc. <doi.org/10.1093/announc/mdmx036≥, 2017; Rugo et al. J. Clin. Oncol. 34:3069, 2016; Senkus et al Annal Onc. 26:v8, 2015; Sareddy & Vadlamudi Clin. J Nat. Med, 13:801, 2015). For early stage breast cancer patients, for example, recommended therapy typically involves tumor resection, followed by ER-targeted therapy (e.g., as discussed below). For advanced breast cancer, including metastatic breast cancer, ER-targeted therapy is the mainstay.

Among other things, presence or development of certain ER mutations has been reported to impact effectiveness of various ER-targeted therapies (see, for example, Jeselsohn et al Nature Rev. Clin. Onc. 12, 573, 2015; Gelsomino et al. Breast Cancer Res. Treat 157:253, 2016; Toy et al. 2013). Some particularly problematic mutations are those that “activate” one or more aspects of ER expression and/or function; some activating mutations have been reported that can render the ER ligand-independent (i.e., constitutively active). For example, particular mutations in the ER ligand binding domain, including D538G and Y537S, have been demonstrated to constitutively activate the ER; other mutations including deletions and/or fusions that remove the ligand binding domain, can have similar effects (see, for example, Li et al. Cell Repts 4:1116, 2013; Veeraraghavan et al Breast Cancer Research and Treatment 158, 219-232, 2016; Veeraraghavan, et al. Nature Comms 5:4577, 2014). Some reports have indicated that as many as 50% of women with metastatic breast cancer may have activating ER mutations detectible in circulating tumor DNA.

Breast cancer therapy, however, is not driven solely by ER-therapy. Classification of a patient as ER+/− and HER2+/− also guides therapy decisions. Loibl and Gianni, The Lancet, 389(10087):2415-2429 (2017). Human epidermal growth factor 2 (HER2) is a transmembrane receptor that belongs to a family of four receptors (EGFR/HER1, HER2, HER3, and HER4) that play a role in regulating cell growth, survival, and differentiation. See Arteaga, et al., Nat. Rev. Clin. Oncol., 9:16-32 (2012). HER2 positivity accounts for about 15-20% of breast cancers. Loibl and Gianni, The Lancet, 389(10087):2415-2429 (2017). As noted by Loibl and Giannai, HER2 overexpression is measured by immunohistochemistry status (IHC3+) or by fluorescence in-situ hybridization (FISH) measurement of a HER2 gene copy number of six or more, or a HER2/CEP17 ratio of 2.0 or greater. Id. HER2 status in a given patient can vary between a primary tumor and a metastases. Id. Continuous inhibition of HER2 signaling is critical for improvement of survival outcomes in metastatic breast cancer. Id.

Nevertheless, regardless of mechanism of action of a particular agent, clinical experience thus far has revealed that incomplete effects (e.g., within an individual patient and/or across patient populations) and/or development of resistance remain a problem.

### Metastatic Disease

Up to 70-80% of patients with metastatic breast cancer die within 5 years. Lim, Cancer Metastasis Rev. (2016) 35, 495-514. The most common metastases in breast cancer are brain, bone, lung, and liver metastases.

For example, brain metastases occur in about 10 to 16% of breast cancer patients, the second most common cause of brain metastases after lung cancer. Leone, Exp Hematol Oncol 4, 33 (2015) doi:10.1186/s40164-015-0028-8. The prognosis is poor, with overall survival from diagnosis ranging from a few months to a few years. Frisk, et al., Breast Cancer Res. Treat. 166:887-896 (2017). In particular, for hormone positive disease e.g., ER-positive breast cancer, the incidence of breast cancer brain metastases (BCBMs) is 14%, with a median overall survival after the development of brain metastases of 9-10 months. Brosnan & Anders, Ann Transl Med., 2016; 6(9):163. Still further, brain metastases occur in 25% to 50% of women having HER2+ breast cancer. Zimmer, et al., Cancer Rep. 2020:e1274 (2020). There are no FDA-approved therapies for treating breast cancer brain metastasis, only surgery and radiotherapy, including whole brain radiotherapy.

The challenge of treating breast cancer brain metastases is providing a therapy that is capable of crossing the blood brain barrier (BBB). As noted by Brosnan & Anders, the BBB exists to selectively regulate what enters the brain and protects it from toxic substances, including chemotherapeutics and targeted drugs. In particular, “the unpredictable nature and heterogeneity in permeability of the BBB, in addition to inherent drug efflux pumps, makes it challenging to effectively deliver drugs in sufficient quantity to brain metastases to achieve apoptosis.” Brosnan & Anders, Ann Transl Med., 2016; 6(9):163.

The present disclosure provides Compound 1, and pharmaceutically acceptable salts thereof, and compositions thereof, that are capable of crossing the blood brain barrier, thereby providing a viable mode of treatment of breast cancer brain metastases, for example in patients who have ER+ and HER2+ disease.

### Complete Estrogen Receptor Antagonists

In some embodiments, the present disclosure teaches particular usefulness of a compound (e.g., Compound 1) that is a complete estrogen receptor antagonist. In some embodiments, a “complete estrogen receptor antagonist,” as that term is used herein, is characterized by complete antagonism of the estrogen receptor with no or minimal residual estrogen receptor agonist activity. For example, it is understood that a complete estrogen antagonist is an agent (e.g., a small molecule compound) that shows ER antagonism and no ER agonism in one or more of ERα protein level assays, MCF-7 cell line assays, Ishikawa cell line assays (measuring wild type ER and certain mutants including mutants lacking AF1 and/or AF2 domains), and rodent uterine weight gain assays. See, generally, WO 2017/059139 and U.S. Pat. No. 9,018,244, each of which is incorporated herein by reference in its entirety. Alternatively or additionally, in some embodiments, a complete estrogen receptor antagonist has three characteristics: it (1) inhibits both activating function 1 (AF1) and activating function 2 (AF2), as complete anti-estrogen activity requires inactivation of both AF1 and AF2; (2) promotes ER degradation; and (3) lacks the partial ER agonist activity observed with certain other agents. Without being bound by theory, it is understood that complete inhibition of both AF1 and AF2 is required for complete estrogen receptor activity, activating mutations in the gene that codes for estrogen receptor 1 allow for activation of both AF1 and AF2 even in the absence of estrogen.

Given the importance of ER signaling in many cancers, as well as in certain cardiovascular, inflammatory, and neurodegenerative diseases, significant effort has been invested in developing therapeutic agents and modalities that target the ER. There is some fluidity/flexibility in terminology that has been used to describe ER-targeting agents, but a variety of agents, with different mechanisms, have been developed and/or studied.

Currently, fulvestrant is the only approved therapy that has each characteristic of a complete estrogen receptor antagonist. But, fulvestrant suffers from numerous shortcomings, including poor oral bioavailability and an inability to cross the blood brain barrier, making it entirely ineffective for the treatment of brain metastases related to ER-associated diseases or disorders.

The present disclosure provides methods of treating a subject suffering from an ER+/HER2+ cancer (e.g., breast cancer). In some embodiments, a method of treating a subject suffering from a cancer that has metastasized to other organs (e.g., the brain, bones, lungs, or liver) comprises administering to the subject a complete estrogen receptor antagonist. In some embodiments, a primary tumor (e.g., a tumor located in a mammary gland of a breast cancer) has the same or different HER2 status as a metastatic lesion.

In some embodiments, a complete estrogen receptor antagonist is Compound 1

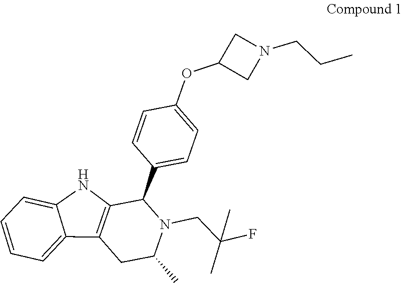 [SMILES: CCCN1CC(Oc2ccc([C@@H]3c4[nH]c5ccccc5c4C[C@@H](C)N3CC(C)(C)F)cc2)C1]

or a pharmaceutically acceptable salt thereof.

Compound 1 is described in WO 2017/059139 as Compound B, otherwise referred to as (1R,3R)-2-(2-fluoro-2-methylpropyl)-3-methyl-1-(4-((1-propylazetidin-3-yl)oxy)phenyl)-2,3,4,9-tetrahydro-1H-pyrido[3,4-b]indole. The synthesis and certain attributes of Compound 1 are reported in WO 2017/059139, which is incorporated herein by reference in its entirety. Certain uses of Compound 1 are also described in WO 2021/007146 and WO 2021/178846, each of which is incorporated herein by reference in its entirety.

### Methods of Use

The present disclosure encompasses the insight that certain complete estrogen receptor antagonists have a number of uses, including treatment of a disease, disorder, or condition (e.g., a cancer associated with ER+ and/or HER2+ status, such as breast cancer, including metastatic brain cancer), detection, and/or characterization of certain tumors.

### Methods of Treatment

For example, in some embodiments, the present disclosure provides certain methods of treatment in a subject having a disease, disorder, or condition, wherein the subject has been determined or is suspected to have an ER+ and HER2+ disease, disorder, or condition. For example, in some embodiments, the present disclosure provides a method of treating an ER-associated disorder in a subject, wherein the subject has been determined to have or is suspected of having metastatic breast cancer (e.g., brain, lung, bone, or liver metastases). In some embodiments, the subject has developed brain metastases related to an ER+ and HER2+ disease, e.g., breast cancer. In some embodiments, the subject has developed bone metastases related to an ER+ and HER2+ disease, e.g., breast cancer. In some embodiments, the subject has developed liver metastases related to an ER+ and HER2+ disease, e.g., breast cancer. In some embodiments, the subject has developed lung metastases related to an ER+ and HER2+ disease, e.g., breast cancer.

In some embodiments, the present disclosure provides a method of treating a metastatic breast cancer in a subject comprising administering to the subject Compound 1, or a pharmaceutically acceptable salt thereof, wherein the subject has previously been treated with an estrogen receptor inhibitor or an aromatase inhibitor. In some embodiments, an estrogen receptor inhibitor is a selective estrogen receptor modulator or a selective estrogen receptor degrader. In some embodiments, an estrogen receptor inhibitor is selected from tamoxifen, endoxifen, raloxifene, toremifene, lasofoxifene, ospemifene, and fulvestrant. In some embodiments, an aromatase inhibitor is selected from letrozole, anastrozole, and exemestane.

In some embodiments, the present disclosure provides, in a method of treating a cancer comprising administering Compound 1 or a pharmaceutically acceptable salt thereof, the improvement comprising administering Compound 1 or a pharmaceutically acceptable salt thereof to a subject that has been determined to have or is suspected of having a ER+/HER2+ cancer. In some such embodiments, an improvement comprises administering the Compound 1 or a pharmaceutically acceptable salt thereof to a subject with metastatic ER+/HER2+ cancer, e.g., cancer that has metastasized to the brain, bones, lung, or liver. In some such embodiments, an improvement comprises administering the Compound 1 or a pharmaceutically acceptable salt thereof to a subject with cancer that has metastasized to the brain.

In some embodiments, the present disclosure provides, in a method of treating cancer in a subject, the improvement that comprises administering to the subject a composition that delivers to the subject's brain Compound 1, wherein the subject has been determined to have a ER+/HER2+ breast cancer and is suspected of having or has been determined to have brain metastases.

In some embodiments, the present disclosure provides, in a method of treating a ER+/HER2+ cancer in a subject with a complete estrogen receptor antagonist, the improvement that comprises administering to the subject the complete estrogen receptor antagonist (e.g., Compound 1 or a pharmaceutically acceptable salt thereof), wherein the subject has been determined or is suspected of having metastases (e.g., in the brain, bones, lungs, or liver). In some embodiments, the present disclosure provides, in a method of treating a ER+/HER2+ cancer in a subject with a complete estrogen receptor antagonist, the improvement that comprises administering to the subject the complete estrogen receptor antagonist (e.g., Compound 1 or a pharmaceutically acceptable salt thereof), wherein the subject has been determined or is suspected of having brain metastases.

In some embodiments, the present disclosure provides a method of treating an ER-associated cancer by: administering to a population of subjects suffering from brain metastases, a composition comprising a complete estrogen receptor antagonist, so that, on average, the brain metastases are reduced or eliminated. In some such embodiments, a subject is suspected of having or has been determined to have a ER+/HER2+ disorder, such as cancer (e.g., breast cancer).

In some embodiments, the present disclosure provides a method of treating an ER-associated cancer by: administering to a subject suffering from brain metastases a composition comprising a complete estrogen receptor antagonist, according to a regimen established, on average, to reduce or eliminate brain metastases in a comparable population of subjects. In some such embodiments, a subject is suspected of having or has been determined to have a ER+/HER2+ disorder, such as cancer (e.g., breast cancer).

In some embodiments, the present disclosure provides a method of treating an ER-associated cancer by: administering to a population of subjects suffering from bone metastases, a composition comprising a complete estrogen receptor antagonist, so that, on average, the bone metastases are reduced or eliminated. In some such embodiments, a subject is suspected of having or has been determined to have a ER+/HER2+ disorder, such as cancer (e.g., breast cancer).

In some embodiments, the present disclosure provides a method of treating an ER-associated cancer by: administering to a subject suffering from bone metastases a composition comprising a complete estrogen receptor antagonist, according to a regimen established, on average, to reduce or eliminate bone metastases in a comparable population of subjects. In some such embodiments, a subject is suspected of having or has been determined to have a ER+/HER2+ disorder, such as cancer (e.g., breast cancer).

In some embodiments, the present disclosure provides a method of treating an ER-associated cancer by: administering to a population of subjects suffering from liver metastases, a composition comprising a complete estrogen receptor antagonist, so that, on average, the liver metastases are reduced or eliminated. In some such embodiments, a subject is suspected of having or has been determined to have a ER+/HER2+ disorder, such as cancer (e.g., breast cancer).

In some embodiments, the present disclosure provides a method of treating an ER-associated cancer by: administering to a subject suffering from liver metastases a composition comprising a complete estrogen receptor antagonist, according to a regimen established, on average, to reduce or eliminate liver metastases in a comparable population of subjects. In some such embodiments, a subject is suspected of having or has been determined to have a ER+/HER2+ disorder, such as cancer (e.g., breast cancer).

In some embodiments, the present disclosure provides a method of treating an ER-associated cancer by: administering to a population of subjects suffering from lung metastases, a composition comprising a complete estrogen receptor antagonist, so that, on average, the lung metastases are reduced or eliminated. In some such embodiments, a subject is suspected of having or has been determined to have a ER+/HER2+ disorder, such as cancer (e.g., breast cancer).

In some embodiments, the present disclosure provides a method of treating an ER-associated cancer by: administering to a subject suffering from lung metastases a composition comprising a complete estrogen receptor antagonist, according to a regimen established, on average, to reduce or eliminate lung metastases in a comparable population of subjects. In some such embodiments, a subject is suspected of having or has been determined to have a ER+/HER2+ disorder, such as cancer (e.g., breast cancer).

In some embodiments, an ER+/HER2+-associated disorder is a cancer. In some embodiments, an ER+/HER2+-associated disorder is a breast cancer. In some embodiments, a subject suffering from an ER+/HER2+-associated disorder has developed a brain, bone, lung, or liver metastasis. In some embodiments, a subject suffering from an ER+/HER2+-associated disorder has developed a brain metastasis.

In some embodiments, the present disclosure provides a method of preventing metastatic spread of an ER+/HER2+ associated cancer to the brain of a subject, the method comprising administering Compound 1:

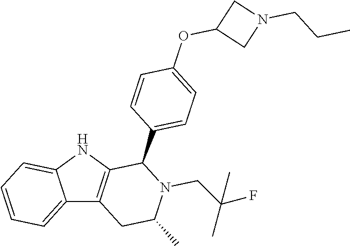 [SMILES: CCCN1CC(Oc2ccc([C@@H]3c4[nH]c5ccccc5c4C[C@@H](C)N3CC(C)(C)F)cc2)C1]

or a pharmaceutically acceptable salt thereof. In some embodiments, preventing metastatic spread of a cancer refers to inhibition of spread of a cancer localized in one part of the subject's body to another organ, e.g., the brain, bones, lung, or liver. In some embodiments, preventing metastatic spread refers to inhibition of spread of a cancer localized in one part of the subject's body to the brain. In some embodiments, provided methods are for preventing metastatic spread of an ER+/HER2+ cancer to another organ, e.g., the brain, bones, lung or liver. In some embodiments, provided methods are for preventing metastatic spread of an ER+/HER2+ cancer to the brain.

### Methods of Detection

The present disclosure further encompasses the insight that certain compounds described herein, e.g., radiolabeled versions of any compound described herein, are useful for detecting certain ER-associated diseases, disorders, and conditions. For example, a compound described herein where a non-radioactive fluorine atom is substituted with ¹⁸F, can be used to detect an ER-associated tumor via, for example PET. In some embodiments, the present disclosure provides a method of detecting an ER-associated disorder in a subject, wherein the subject has been determined or is suspected of having brain metastases. That is, in some embodiments, the present disclosure provides a method of detecting an ER-associated metastases in the brain. In some embodiments, compounds described herein, e.g., wherein a non-radioactive fluorine atom is substituted with ¹⁸F, can be used to detect ER-associated metastases in the brain in a subject suffering from a ER+/HER2+ cancer (e.g., breast cancer).

### Methods of Characterizing

The present disclosure further encompasses the insight that efficacy of certain compounds described herein is assessed by in vitro and in vivo model assays. For example, certain compounds described herein are characterized according to any of the model assays described herein, including, for example, ERα protein level assays, MCF-7 cell line assays, Ishikawa cell line assays, ECC-1 cell line assays (measuring wild type ER and certain mutants including mutants lacking AF1 and/or AF2 domains), rodent uterine weight gain assays, BT-474 cell line assays, ZR-75-1 cell line assays, or ZR-75-30 cell line assays. Further, in some embodiments, the compounds described herein, when characterized by any referenced assay, exhibit complete estrogen receptor antagonism, as that term has been defined herein.

### Dosing

In some embodiments, the present disclosure provides methods of treating a subject suspected of having or determined to have a ER+/HER2+ disorder (e.g., cancer, e.g., breast cancer), comprising administering a composition comprising a complete estrogen receptor antagonist (e.g., Compound 1 or a pharmaceutically acceptable salt thereof). In some embodiments, the present disclosure provides a method of treating a subject suffering from an ER-associated disorder (e.g., a ER+/HER2+ disorder), wherein the subject has developed or is suspected to have developed brain metastases, the method comprising administering a composition comprising a complete estrogen receptor antagonist. In some embodiments, the composition comprises a complete estrogen receptor antagonist and a pharmaceutically acceptable excipient, carrier, or diluent. Said composition may be administered orally, parenterally, by inhalation or nasal spray, topically (e.g., as by powders, ointments, or drops), rectally, buccally, intravaginally, intraperitoneally, intracisternally or via an implanted reservoir, depending on the severity of the condition being treated. Preferably, the compositions are administered orally, intraperitoneally or intravenously. In certain embodiments, provided compounds are administered orally or parenterally at dosage levels of about 0.01 mg/kg to about 50 mg/kg, of subject body weight per day, one or more times a day, to obtain the desired therapeutic effect. In some embodiments, provided compounds are administered orally or parenterally at dosage levels of about 0.05 mg/kg to about 5 mg/kg, of subject body weight per day, one or more times a day, to obtain the desired therapeutic effect.

Pharmaceutically acceptable compositions described herein may be orally administered in any orally acceptable dosage form including, but not limited to, capsules, tablets, aqueous suspensions or solutions. In such solid dosage forms, the active compound may be admixed with at least one inert diluent such as sucrose, lactose, or starch. Such dosage forms may also comprise, as is normal practice, additional substances other than inert diluents, e.g., lubricants and other tableting aids such a magnesium stearate and microcrystalline cellulose. When aqueous suspensions are required for oral use, the active ingredient is combined with emulsifying and suspending agents. If desired, certain sweetening, flavoring, or coloring agents may also be added.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and/or i) lubricants such as talc, calcium stearate, magnesium stearate, solid polyethylene glycols, sodium lauryl sulfate, and mixtures thereof. In the case of capsules, tablets and pills, the dosage form may also comprise buffering agents. The active compounds can also be in micro-encapsulated form with one or more excipients as noted above.

Solid compositions of a similar type may also be employed as fillers in soft and hard-filled gelatin capsules using such excipients as lactose or milk sugar, as well as high molecular weight polyethylene glycols and the like. The solid dosage forms of tablets, dragees, capsules, pills, and granules can be prepared with coatings and shells such as enteric coatings (i.e. buffering agents) and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Alternatively, pharmaceutically acceptable compositions described herein may be administered in the form of suppositories for rectal or vaginal administration. These can be prepared by mixing the compounds of the present application with suitable non-irritating excipients or carriers that are solid at room temperature but liquid at body (e.g. rectal or vaginal) temperature and therefore will melt in the rectum or vaginal cavity to release the active compound. Such materials include cocoa butter, a suppository wax (e.g., beeswax) and polyethylene glycols.

A person of skill in the art would readily understand how a therapeutically effective dose determined for an animal can be converted to the corresponding human equivalent dose. Accordingly, a person of skill in the art would understand that certain provided data for an animal (e.g., a mouse) can be used to determine a suitable dose in a human, for example by using the table provided by Nair & Jacob, J. Basic Clin. Pharm., 7(2):27-31 (2016).

The present disclosure provides dosing regimens where compounds reported herein are dosed at levels and/or according to regimens corresponding to those exemplified herein for Compound 1 (see, e.g., Example 4). That is, a dose (i.e., a composition optionally comprising additional pharmaceutically acceptable excipients) refers to a particular ratio of compound weight per kilogram of subject. For example, a dose of 3 mg/kg refers to a composition, optionally comprising pharmaceutically acceptable excipients, where the compound is administered to the subject in an amount that is 3 milligrams for every kilogram of subject weight. It is understood that a weight of a compound is determined according to the free base weight of a compound (e.g., if a compound is a salt, the corresponding free base weight of the compound is used to determine the amount of compound in the dose). Accordingly, in some embodiments, a human subject is provided a dose that corresponds to a 3 mg/kg to 30 mg/kg in a mouse. In some embodiments, a human subject is provided a dose that corresponds to 3, 4, 5, 6, 7, 8, 9, 10, 11, 12, 13, 14, 15, 16, 17, 18, 19, 20, 21, 22, 23, 24, 25, 26, 27, 28, 29, or 30 mg/kg in a mouse. In some embodiments, a human subject is provided a dose that corresponds to greater than or equal to 3 mg/kg in a mouse. In some embodiments, a human subject is provided a dose that corresponds to greater than or equal to 5 mg/kg in a mouse. In some embodiments, a human subject is provided a dose that corresponds to greater than or equal to 10 mg/kg in a mouse. In some embodiments, a human subject is provided a dose that corresponds to greater than or equal to 15 mg/kg in a mouse. In some embodiments, a human subject is provided a dose that corresponds to greater than or equal to 20 mg/kg in a mouse. In some embodiments, a human subject is provided a dose that corresponds to greater than or equal to 25 mg/kg in a mouse. In some embodiments, a human subject is provided a dose that corresponds to greater than or equal to 30 mg/kg in a mouse.

In some embodiments, a composition comprising a complete estrogen receptor antagonist is administered as a unit dosage form. In some embodiments, a composition comprising a complete estrogen receptor antagonist is administered in the form of a capsule. In some embodiments, a composition comprising a complete estrogen receptor antagonist is administered in the form of a tablet. In some embodiments, a composition comprising a complete estrogen receptor antagonist is administered as a suspension. In some embodiments, a composition comprising a complete estrogen receptor antagonist is administered as a solution.

In some embodiments, a composition comprising a complete estrogen receptor antagonist is administered as a daily dose (QD). In some embodiments, a composition comprising a complete estrogen receptor antagonist is administered as a twice daily dose (BID). In some embodiments, a composition comprising a complete estrogen receptor antagonist is administered every other day (QOD). In some embodiments, a composition comprising a complete estrogen receptor antagonist is administered as a weekly dose (QW). In some embodiments, a composition comprising a complete estrogen receptor antagonist is administered as a monthly dose (Q4W).

In some embodiments, Compound 1 is administered to the subject in an amount that is from about to 15 mg to about 360 mg. In some embodiments, Compound 1 is administered to the subject in an amount that is from about to 30 mg to about 360 mg. In some embodiments, Compound 1 is administered to the subject in an amount that is from about to 30 mg to about 300 mg. In some embodiments, Compound 1 is administered to the subject in an amount that is from about to 60 mg to about 120 mg. In some embodiments, Compound 1 is administered to the subject in an amount that is from about 15 mg to about 100 mg. In some embodiments, Compound 1 is administered to the subject in an amount that is about 15 mg, about 20 mg, about 30 mg, about 40 mg, about 50 mg, about 60 mg, about 70 mg, about 80 mg, about 90 mg, or about 100 mg. In some embodiments, Compound 1 is administered to the subject in an amount that is about 120 mg, about 150 mg, about 210 mg, or about 300 mg. In some embodiments, Compound 1 is administered to the subject in an amount that is about 30 mg. In some embodiments, Compound 1 is administered to the subject in an amount that is about 60 mg. In some embodiments, Compound 1 is administered to the subject in an amount that is about 90 mg. In some embodiments, Compound 1 is administered to the subject in an amount that is about 120 mg.

In some embodiments, Compound 1 is administered to the subject in an amount that is about 15 mg to about 360 mg per day (QD). In some embodiments, Compound 1 is administered to the subject in an amount that is about 30 mg to about 360 mg per day (QD). In some embodiments, Compound 1 is administered to the subject in an amount that is about 30 mg to about 300 mg per day (QD). In some embodiments, Compound 1 is administered to the subject in an amount that is about 60 mg to about 120 mg per day (QD). In some embodiments, Compound 1 is administered to the subject in an amount that is from about 15 mg to about 100 mg QD. In some embodiments, Compound 1 is administered to the subject in an amount that is about 15 mg, about 20 mg, about 30 mg, about 40 mg, about 50 mg, about 60 mg, about 70 mg, about 80 mg, about 90 mg, or about 100 mg QD. In some embodiments, Compound 1 is administered to the subject in an amount that is about 120 mg, about 150 mg, about 210 mg, or about 300 mg QD. In some embodiments, Compound 1 is administered to the subject in an amount that is about 30 mg QD. In some embodiments, Compound 1 is administered to the subject in an amount that is about 60 mg QD. In some embodiments, Compound 1 is administered to the subject in an amount that is about 90 mg QD. In some embodiments, Compound 1 is administered to the subject in an amount that is about 120 mg QD.

In some embodiments, Compound 1 is administered to the subject in a unit dosage form. In some embodiments, unit dosage form is a capsule or tablet. In some embodiments, a unit dosage form comprises about 15 mg to about 120 mg of Compound 1. In some embodiments, a unit dosage form comprises about 15 mg to about 100 mg of Compound 1. In some embodiments, a unit dosage form comprises about 60 mg to about 120 mg of Compound 1. In some embodiments, a unit dosage form comprises about 15 mg, about 20 mg, about 30 mg, about 40 mg, about 50 mg, about 60 mg, about 70 mg, about 80 mg, about 90 mg, or about 100 mg of Compound 1. In some embodiments, a unit dosage form comprises about 15 mg of Compound 1. In some embodiments, a unit dosage form comprises about 30 mg of Compound 1. In some embodiments, a unit dosage form comprises about 60 mg of Compound 1. In some embodiments, a unit dosage form comprises about 90 mg of Compound 1. In some embodiments, a unit dosage form comprises about 120 mg of Compound 1. In some embodiments, a unit dosage form is a capsule. In some embodiments, a unit dosage form is a tablet.

In some embodiments, a total daily dose of Compound 1 administered to the subject is in an amount that is about 15 mg to about 360 mg per day (QD). In some embodiments, a total daily dose of Compound 1 administered to the subject is about 30 mg to about 360 mg. In some embodiments, a total daily dose of Compound 1 administered to the subject is about 30 mg to about 300 mg. In some embodiments, a total daily dose of Compound 1 administered to the subject is about 60 mg to about 120 mg. In some embodiments, a total daily dose of Compound 1 administered to the subject is in an amount that is from about 15 mg to about 100 mg QD. In some embodiments, a total daily dose of Compound 1 administered to the subject is in an amount that is about 15 mg, about 20 mg, about 30 mg, about 40 mg, about 50 mg, about 60 mg, about 70 mg, about 80 mg, about 90 mg, or about 100 mg QD. In some embodiments, a total daily dose of Compound 1 administered to the subject is about 120 mg, about 150 mg, about 210 mg, or about 300 mg. In some embodiments, a total daily dose of Compound 1 administered to the subject is in an amount that is about 30 mg QD. In some embodiments, a total daily dose of Compound 1 administered to the subject is about 60 mg. In some embodiments, a total daily dose of Compound 1 administered to the subject is about 90 mg. In some embodiments, a total daily dose of Compound 1 administered to the subject is about 120 mg.

The present disclosure also encompasses the recognition that Compound 1 advantageously can be used to treat metastasized cancers, e.g., cancers that have spread to the brain, bones, lungs, liver, or the central nervous system. As illustrated the table below, Compound 1, when administered in a single oral 10 or 30 mg/kg dose, is able to penetrate the blood brain barrier. Other estrogen receptor antagonists, e.g., fulvestrant, are unable to penetrate the blood-brain barrier in analogous quantities.

| | | | Dose | Concentration |
|---|---|---|---|---|
| Matrix | Group | Dose | Unit | (ng/ml) |
| Plasma | Compound 1, 10 mpk | 10 | mg/kg | 1919 |
| Plasma | Compound 1, 30 mpk | 30 | mg/kg | 5558 |
| Brain | Compound 1, 10 mpk | 10 | mg/kg | 2147 |
| Brain | Compound 1, 30 mpk | 30 | mg/kg | 9708 |
| Brain | Fulvestrant, 5 mg qw | 5 | mg | 501 |

mpk = mg/kg;
qw = once weekly

### Combination Therapies

The present disclosure encompasses the recognition that a combination of certain agents can beneficially be used to completely antagonize the estrogen receptor. Accordingly, in some embodiments, the present disclosure provides a method of treating a subject suffering from an ER-associated disorder (e.g., a cancer or breast cancer, e.g., a ER+/HER2+ cancer or breast cancer) comprising administering a complete estrogen receptor antagonist (e.g., Compound 1, or a pharmaceutically acceptable salt thereof) and an anti-cancer agent. For example, in some embodiments, the anti-cancer agent is a CDK 4/6 inhibitor, a PI3Kalpha inhibitor, an mTOR inhibitor, or a HER2 inhibitor.

In some embodiments, the present disclosure provides a method of treating a patient or subject suffering from a cancer, the method comprising administering a complete estrogen receptor antagonist (e.g., Compound 1, or a pharmaceutically acceptable salt thereof) and an anti-cancer agent is a CDK4/6 inhibitor (i.e., inhibits one or both of CDK4 and CDK6). In some embodiments, an anti-cancer agent is a CDK4/6 inhibitor selected from palbociclib, ribociclib, abemaciclib, lerociclib, trilaciclib, and SHR6390. In some embodiments, the CDK 4/6 inhibitor is palbociclib. In some embodiments, the CDK4/6 inhibitor is ribociclib. In some embodiments, the CDK4/6 inhibitor is abemaciclib. In some embodiments, the CDK4/6 inhibitor is lerociclib. In some embodiments, the CDK4/6 inhibitor is trilaciclib. In some embodiments, the CDK 4/6 inhibitor is SHR6390.

In some embodiments, the present disclosure provides a method of treating a patient or subject suffering from a cancer, the method comprising administering a complete estrogen receptor antagonist (e.g., Compound 1, or a pharmaceutically acceptable salt thereof) and an anti-cancer agent, wherein the secondary agent is a PI3Kalpha inhibitor. In some embodiments, the PI3Kalpha inhibitor is selected from alpelisib, taselisib, and LY3023414. In some embodiments, the PI3Kalpha inhibitor is alpelisib. In some embodiments, the PI3K alpha inhibitor is taselisib. In some embodiments, the PI3Kalpha inhibitor is LY3023414.

In some embodiments, the present disclosure provides a method of treating a patient or subject suffering from a cancer, the method comprising administering a complete estrogen receptor antagonist (e.g., Compound 1, or a pharmaceutically acceptable salt thereof) and an anti-cancer agent, wherein an anti-cancer agent is an mTOR inhibitor. In some embodiments, the mTOR inhibitor is selected from sirolimus, temsirolimus, everolimus, and LY3023414. In some embodiments, the mTOR inhibitor is sirolimus. In some embodiments, the mTOR inhibitor is temsirolimus. In some embodiments, the mTOR inhibitor is everolimus. In some embodiments, the mTOR inhibitor is LY3023414.

In some embodiments, the present disclosure provides a method of treating a patient or subject suffering from a cancer, the method comprising administering a complete estrogen receptor antagonist (e.g., Compound 1, or a pharmaceutically acceptable salt thereof) and an anti-cancer agent, wherein an anti-cancer agent is a HER2 inhibitor. In some embodiments, a HER2 inhibitor is tucatinib, trastuzumab, ado-trastuzumab emtansine, trastuzumab deruxtecan, pertuzumab, lapatinib, or neratinib. In some embodiments, a HER2 inhibitor is tucatinib. In some embodiments, a HER2 inhibitor is trastuzumab. In some embodiments, a HER2 inhibitor is ado-trastuzumab emtansine. In some embodiments, a HER2 inhibitor is trastuzumab deruxtecan. In some embodiments, a HER2 inhibitor is pertuzumab. In some embodiments, a HER2 inhibitor is lapatinib. In some embodiments, a HER2 inhibitor is neratinib.

In some embodiments, a HER2 inhibitor is trastuzumab and is administered in a dose of about 2 mg/kg to about 8 mg/kg (e.g., 2 mg/kg, 4 mg/kg, 6 mg/kg, or 8 mg/kg). In some embodiments, trastuzumab is administered weekly. In some embodiments, trastuzumab is administered once every three weeks. In some embodiments, trastuzumab is administered in an initial dose of 4 mg/kg, then in a dose of 2 mg/kg weekly for a period of time, e.g., 52 weeks. In some embodiments, trastuzumab is administered in an initial dose of 8 mg/kg, then in a dose of 6 mg/kg every three weeks for a period of time, e.g., 52 weeks. In some embodiments, trastuzumab is administered as an intravenous infusion.

In some embodiments, a HER2 inhibitor is ado-trastuzumab emtansine, and is administered at a dose of about 3.6 mg/kg once every three weeks. In some embodiments, a HER2 inhibitor is ado-trastuzumab emtansine, and is administered at a dose of about 100 mg to about 160 mg (e.g., about 100 mg or about 160 mg) once every three weeks. In some embodiments, ado-trastuzumab emtansine is administered as an intravenous infusion.

In some embodiments, a HER2 inhibitor is trastuzumab deruxtecan, and is administered at a dose of about 5.4 mg/kg to about 6.4 mg/kg once every three weeks. In some embodiments, trastuzumab deruxtecan is administered in a dose of about 5.4 mg/kg every three weeks. In some embodiments, trastuzumab deruxtecan is administered in a dose of about 6.4 mg/kg every three weeks. In some embodiments, a HER2 inhibitor is trastuzumab deruxtecan, and is administered at a dose of about 100 mg once every three weeks. In some embodiments, trastuzumab deruxtecan is administered as an intravenous infusion.

In some embodiments, the present disclosure provides a method of treating a patient or subject suffering from a cancer, the method comprising administering a complete estrogen receptor (e.g., Compound 1, or a pharmaceutically acceptable salt thereof) antagonist and two or more anti-cancer agents. In some embodiments, each of the two or more anti-cancer agents are selected from a CDK 4/6 inhibitor, a PI3Kalpha inhibitor, an mTOR inhibitor, and a HER2 inhibitor. In some embodiments, one anti-cancer agent is a CDK 4/6 inhibitor, and the other anti-cancer agent is a HER2 inhibitor. In some embodiments, one anti-cancer agent is ribociclib, abemaciclib, or palbociclib, and the other anti-cancer agent is tucatinib. In some embodiments, one anti-cancer agent is ribociclib, and the other anti-cancer agent is tucatinib. In some embodiments, both anti-cancer agents are HER2 inhibitors. In some embodiments, one anti-cancer agent is tucatinib, and the other anti-cancer agent is trastuzumab. In some embodiments, one anti-cancer agent is tucatinib, and the other anti-cancer agent is ado-trastuzumab emtansine. In some embodiments, one anti-cancer agent is tucatinib, and the other anti-cancer agent is trastuzumab deruxtecan. In some embodiments, one anti-cancer agent is trastuzumab, and the other anti-cancer agent is pertuzumab.

It is understood that administering a complete estrogen receptor antagonist and one or more anti-cancer agents described herein can be administered simultaneously or separately. For example, in some embodiments, a complete estrogen receptor antagonist and an anti-cancer agent are administered simultaneously. In some embodiments, an anti-cancer agent is administered prior to administration of a complete estrogen receptor antagonist. In some embodiments, an anti-cancer agent is administered after administration of a complete estrogen receptor antagonist.

### EXEMPLARY EMBODIMENTS

Embodiment 1. A method of treating cancer in a subject determined to have an ER+ and HER2+ cancer, the method comprising a step of:
   administering to the subject Compound 1:

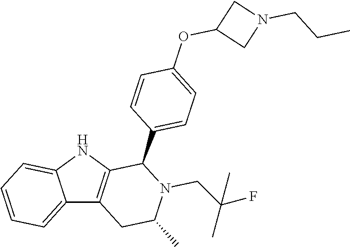 [SMILES: CCCN1CC(Oc2ccc([C@@H]3c4[nH]c5ccccc5c4C[C@@H](C)N3CC(C)(C)F)cc2)C1]

or a pharmaceutically acceptable salt thereof.
Embodiment 2. The method of Embodiment 1, wherein the subject has been determined to have or is suspected of having brain metastases.
Embodiment 3. The method of Embodiment 2, wherein the HER2 status of a primary tumor associated with breast cancer and a brain metastases is the same.
Embodiment 4. The method of Embodiment 2, wherein the HER2 status of a primary tumor associated with breast cancer and a brain metastases is the different.
Embodiment 5. The method of any one of Embodiments 1-4, wherein the method further comprises administering one or more anti-cancer agents.
Embodiment 6. The method of Embodiment 5, wherein the anti-cancer agent is a CDK 4/6 inhibitor, a PI3Kalpha inhibitor, an mTOR inhibitor, or an HER2 inhibitor.
Embodiment 7. The method of Embodiment 6, wherein the anti-cancer agent is a CDK4/6 inhibitor.
Embodiment 8. The method of Embodiment 7, wherein the CDK4/6 inhibitor is selected from palbociclib, ribociclib, abemaciclib, lerociclib, and trilaciclib.
Embodiment 9. The method of Embodiment 8, wherein the CDK4/6 inhibitor is selected from ribociclib, palbociclib, and abemaciclib.
Embodiment 10. The method of Embodiment 9, wherein the CDK4/6 inhibitor is ribociclib.
Embodiment 11. The method of Embodiment 6, wherein the anti-cancer agent is a PI3Kalpha inhibitor.
Embodiment 12. The method of Embodiment 11, wherein the PI3Kalpha inhibitor is selected from alpelisib and taselisib.
Embodiment 13. The method of Embodiment 6, wherein the anti-cancer agent is an mTOR inhibitor.
Embodiment 14. The method of Embodiment 13, wherein the mTOR inhibitor is selected from sirolimus, temsirolimus, and everolimus.
Embodiment 15. The method of Embodiment 6, wherein the anti-cancer agent is a HER2 inhibitor.
Embodiment 16. The method of Embodiment 15, wherein the HER2 inhibitor is selected from tucatinib, trastuzumab, ado-trastuzumab emtansine, trastuzumab deruxtecan, pertuzumab, lapatinib, or neratinib.
Embodiment 17. The method of Embodiment 16, wherein the HER2 inhibitor is tucatinib.
Embodiment 18. The method of Embodiment 5, comprising administering two or more anti-cancer agents.
Embodiment 19. The method of Embodiment 18, wherein one anti-cancer agent is a CDK4/6 inhibitor, and the other anti-cancer agent is a HER2 inhibitor.
Embodiment 20. The method of any one of Embodiments 1-19, wherein the subject has previously been treated with an estrogen receptor inhibitor or an aromatase inhibitor.
Embodiment 21. The method of Embodiment 20, wherein the estrogen receptor inhibitor is a selective estrogen receptor modulator or a selective estrogen receptor degrader.
Embodiment 22. The method of Embodiment 21, wherein the estrogen receptor inhibitor is selected from tamoxifen, endoxifene, raloxifene, toremifene, lasofoxifene, ospemifene, fulvestrant.
Embodiment 23. The method of Embodiment 20, wherein the aromatase inhibitor is selected from letrozole, anastrozole, and exemestane.
Embodiment 24. In a method of treating cancer in a subject determined to have an ER+ and HER2+ cancer, the improvement comprising administering to the subject Compound 1:

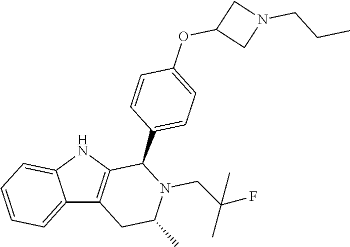 [SMILES: CCCN1CC(Oc2ccc([C@@H]3c4[nH]c5ccccc5c4C[C@@H](C)N3CC(C)(C)F)cc2)C1]

or a pharmaceutically acceptable salt thereof.

### EXEMPLIFICATION

The Examples provided herein document and support certain aspects of the present disclosure but are not intended to limit the scope of any claim. Unless specifically presented in the past tense, inclusion in the Examples is not intended to imply that work described has been completed, or even performed. The following non-limiting examples are provided to further illustrate certain teachings provided by the present disclosure. Those of skill in the art, in light of the present application, will appreciate that various changes can be made in the specific embodiments that are illustrated in the present Examples without departing from the spirit and scope of the present teachings.

The following abbreviations may be used in the Examples below: aq. (aqueous); ACN (acetonitrile); CSA (camphorsulfonic acid); d (day or days); DCM (dichloromethane); DEA (diethylamine); DHP (dihydropyran); DMF (N,N-dimethylformamide); DIPEA (N,N-diisopropylethylamine); DMAP (4-dimethylaminopyridine); DMSO (dimethyl sulphoxide); EA (ethyl acetate); ee (enantiomeric excess); equiv. (equivalent); ethanol (EtOH); h (hour or hours); Hex (hexanes); HPLC (high-performance liquid chromatography); IPA (isopropyl alcohol); KHMDS (potassium bis(trimethylsilyl)amide); LAH (lithium aluminum hydride); LCMS (liquid chromatography-mass spectrometry); LDA (lithium diisopropylamide); LiHMDS (lithium bis(trimethylsilyl)amide); MeOH (methanol); min (minute or minutes); NMR (nuclear magnetic resonance); Pd/C (palladium on carbon); PPh₃O (triphenylphosphine oxide); Pt/C (platinum on carbon); rb (round-bottomed); Rf (retention factor); rt or RT (room temperature); SM (starting material); TEA (triethylamine); THE (tetrahydrofuran); THP (tetrahydropyran); TLC (thin layer chromatography); TsOH (p-toluenesulfonic acid or tosylic acid); and UV (ultraviolet).

### Example 1: Synthesis of Compound 1

The complete synthesis of Compound 1 is provided in PCT App. Pub. No. WO 2017/059139 (referred to as Compound B, or, (1R,3R)-2-(2-fluoro-2-methylpropyl)-3-methyl-1-(4-((1-propylazetidin-3-yl)oxy)phenyl)-2,3,4,9-tetrahydro-1H-pyrido[3,4-b]indole), which is incorporated herein by reference and repeated below.

### Preparation of 4-((1-propylazetidin-3-yl)oxy)benzaldehyde

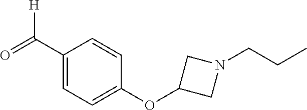 [SMILES: CCCN1CC(Oc2ccc(C=O)cc2)C1]

### Step 1: Preparation of 1-propionylazetidin-3-one

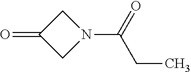

The compound 3-azetidinone hydrochloride (10.000 g, 93.0 mmol, 1.0 equiv.), anhydrous 1,2-dichloroethane (200 mL) and diisopropylethylamine (38.9 mL, 223 mmol, 2.4 equiv.) were added to a round bottom flask (500 mL) to provide a light yellow suspension. The suspension was sonicated for 1 h and then cooled to −10° C. (dry-ice/MeOH) for 10 min. Propionyl chloride (9.8 mL, 112 mmol, 1.2 equiv.) was added dropwise to the cooled suspension to provide an orange solution. The reaction was removed from the bath and stirred at room temperature for 16 h. The solvent was removed to provide a semi-solid. The semi-solid was suspended into EA (300 mL) and the suspension was filtered. The solid was rinsed with EA (2×100 mL). TLC analysis (10% MeOH/DCM, KMnO₇ stain/Heat) indicated there were three spots: Rf: 0.2, 0.5, 0.7. TLC (50% EA/Hex, KMnO₇ stain/Heat) indicated there were two spots: Rf: 1, 0.3. The filtrate was concentrated, adsorbed onto silica gel (25 g) and chromatographed through silica gel (100 g cartridge) with DCM (5 min) then 0-10% MeOH over 15 min. The product came off early from the column in DCM and continued to elute from the column with up to 10% MeOH. TLC in both solvent systems was carried out to determine if any propionyl chloride was present in early fractions. Fractions containing product were pooled and concentrated to afford the title compound as a yellow liquid (11.610 g, 98.2%).

¹H NMR (300 MHz, CDCl₃) δ: 4.80 (d, J=5.6 Hz, 4H), 2.29 (q, J=7.5 Hz, 2H), 2.01 (s, 3H), 1.18 (t, J=7.5 Hz, 3H).

### Step 2. Preparation of 1-propylazetidin-3-ol

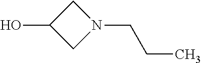

Lithium aluminum hydride (10.397 g, 273.9 mmol, 3.0 equiv.) was suspended into THE (200 mL) and cooled in an ice bath. A solution of 1-propionylazetidin-3-one (11.610 g, 91.3 mmol, 1.0 equiv.) in THE (100 mL) was added dropwise to the reaction mixture via a pressure equalizing addition funnel over 30 min. The addition funnel was removed. The flask was then fitted with a condenser and the reaction was heated at reflux in an oil bath at 75° C. for 16 h. The reaction was cooled in an ice bath for 20 min and sodium sulfate decahydrate (Glauber's salt, 25 g) was added in small portions over 20 min. After complete addition, the mixture was stirred at room temperature for 2 h. The mixture was filtered through a bed of Celite© (2 cm) and the solids rinsed with EA (2×250 mL). The clear solution was concentrated to a pale yellow liquid (9.580 g, 91.1%). NMR indicated the presence of THE and EA. This material was used without further purification in the preparation of the compounds of the examples below.

¹H NMR (300 MHz, CDCl₃) δ: 4.39 (pent, J=6 Hz, 1H), 3.62-3.56 (m, 2H), 2.90-2.85 (m, 2H), 2.41 (t, J=7.5 Hz, 2H), 1.34 (hextet, J=7.2 Hz, 2H), 0.87 (t, J 7.8 Hz, 3H).

### Step 3. Preparation of 4-((1-propylazetidin-3-yl)oxy)benzaldehyde

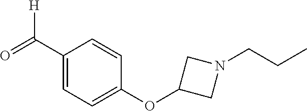 [SMILES: CCCN1CC(Oc2ccc(C=O)cc2)C1]

4-Fluorobenzaldehyde (15.00 g, 120.9 mmol, 0.9 equiv.), 1-propylazetidin-3-ol (15.00 g, 130.2 mmol, 1.0 equiv.), cesium carbonate (88.40 g, 271.3 mmol, 2.1 equiv.) and N,N-dimethylformamide (284 mL) were mixed together with a Teflon™ stir bar in a 500 mL round bottomed flask. The flask was sealed and heated in a heat block at 95° C. for 6 h. The reaction was analyzed by LCMS to indicate the aldehyde was consumed. The suspension was filtered through a sintered glass funnel and the solid was washed with ethyl acetate (100 mL). The filtrate was concentrated to an orange suspension. The suspension was mixed with water (200 mL) and ethyl acetate (200 mL) and the organic layer was washed with water (3×200 mL), brine, dried over anhydrous magnesium sulfate, filtered and concentrated to an orange liquid (21.74 g, 76.1%). The material was used without further purification.

¹HNMR (300 MHz, CDCl₃), δ 9.87 (s, 1H), 7.82 (d, J=9.0 Hz, 2H), 6.86 (d, J=8.7 Hz, 2H), 4.86 (quintet, J=5.7 Hz, 1H), 3.85-3.80 (m, 2H), 3.13-3.08 (m, 2H), 2.48 (t, J=7.2 Hz, 2H), 1.46-1.34 (m, 2H), 0.91 (t, J=7.2 Hz, 3H).

### Preparation of (R)-1-(1H-indol-3-yl)-N—((R)-1-phenylethyl)propan-2-amine

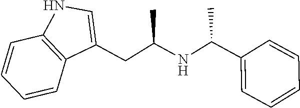 [SMILES: C[C@H](Cc1c[nH]c2ccccc12)N[C@H](C)c1ccccc1]

Indole-3-acetone (25.0 g, 144 mmol, 1.0 equiv.) was added to a solution of (R)-(+)-1-phenylethylamine (23.0 mL, 181 mmol, 1.3 equiv.) in dichloromethane (600 mL) under N₂ at 25° C. and the mixture was allowed to stir for 1 hr. The reaction was cooled to 0-5° C. and sodium triacetoxyborohydride (100 g, 472 mmol, 3.3 equiv.) was added over 30 minutes via powder addition funnel to the ice cooled solution. The orange solution was stirred for 1 h at 0° C. and then was allowed to warm to RT. The reaction was stirred at RT for 19 h. At this time, ESI+ indicated that no indole starting material was present. Saturated NaHCO₃ solution (100 mL) was added in 5 mL portions over 15 min at 10° C. with vigorous stirring. The solution was stirred for 15 min and sat. Na₂CO₃ solution (200 mL) was added over 15 minutes. Solid K₂CO₃ (9 g) was added in 3 g portions at which point the aqueous layer was pH 12 and bubbles had stopped forming. The layers were filtered and separated. The red organic layer was washed with sat. aq. NaHCO₃ (2×100 mL). The aqueous layers were combined and extracted with DCM (2×100 mL). The combined organic layers were dried over Na₂SO₄, filtered and concentrated to give the crude product (49 g). TLC (90:10 DCM:MeOH) showed four spots (Rf=0.63, 0.50, 0.16, 0.26), two of which were the separated diastereomeric major products (Rf=0.16 and 0.26). The crude was adsorbed onto silica gel and purified via flash chromatography (330 g cartridge, 0-100% EA:Hex). Fractions containing the R,R diastereomer were pooled and purified a second time with the same flash chromatography conditions to afford 24 g of product (˜82% ee). Previous successful separation was achieved by a silica gel:crude ratio of 40:1, so the mixture was divided into 3 portions and separated on 3×330 g silica gel cartridges (0-40% EA/Hex for 20 min, isocratic 40% EA/Hex 40 min). All fractions containing the desired product were >99% diastereomerically pure. Pure fractions were concentrated and pooled to yield (R)-1-(1H-indol-3-yl)-N—((R)-1-phenylethyl)-propan-2-amine as an orange semi-solid (11.91 g, 29.6%).

¹H NMR (CDCl₃, 300 MHz) R,R diastereomer: δ 0.96 (d, J=6.6 Hz, 3H), 1.30 (d, J=6.6 Hz, 3H), 2.68 (q, J=7.2 Hz, 1H), 2.97 (m, 2H) 4.00 (q, J=6.3 Hz, 1H), 7.43-6.97 (m, 10H), 7.96 (br s, 1H). R,S diastereomer: δ 1.11 (d, J=5.7 Hz, 3H), 1.30 (d, J=5.4 Hz, 3H) 2.80 (m, 3H), 3.92 (q, J=6.9 Hz, 1H), 6.93-7.40 (m, 10H), 8.13 (br s, 1H); the aromatic region was difficult to distinguish from the R,R diastereomer due to lack of purity.

LCMS: ES+ [M+H]+ 279.0.

### Preparation of (2R)-1-(1H-indol-3-yl)propan-2-amine

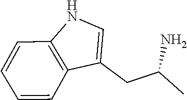

The compound (R)-1-(1H-indol-3-yl)-N—((R)-1-phenylethyl)propan-2-amine (11.91 g, 42.8 mmol, 1.0 equiv.) was dissolved in methanol (250 mL) and added to a 2 L Parr bottle and the solution was sparged with N₂ for 10 min. 20% Pd(OH)₂ on carbon wet with water (10.71 g, 76.3 mmol, 1.8 equiv.) was added and the bottle was pressurized with 50 psi of hydrogen and shaken in a Parr apparatus for 22 h, LCMS analysis indicated that the reaction was completed. The suspension was filtered through Celite® and concentrated to remove MeOH. The crude was dissolved into DCM and washed with saturated Na₂CO₃ solution (50 mL) and the aqueous layer was extracted with DCM (2×50 mL). The organic layers were combined, dried, and concentrated to yield (2R)-1-(1H-indol-3-yl)propan-2-amine as a light brown solid that did not require further purification (6.68 g, 89.6%).

¹H NMR (CDCl₃, 300 MHz) δ 1.17 (d, J=6.6 Hz, 3H), 2.66 (dd, J=8.4, 14.7 Hz, 1H), 2.88 (dd, J=5.4, 14.1 Hz, 1H), 3.27 (sextet, J=1.5 Hz, 1H), 7.05-7.22 (m, 3H), 7.37 (d, J=7.5 Hz, 1H), 7.62 (d, J=8.7 Hz, 1H), 8.00 (br s, 1H).

LCMS: ES+ [M+H]+ 174.9.

### Preparation of 2-fluoro-2-methylpropanol

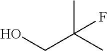

Methyl 2-fluoro-2-methylpropionate (5.01 g, 40.5 mmol, 1.0 equiv.) was added dropwise over 15 min to a stirred suspension of lithium aluminum hydride (2.50 g, 65.9 mmol, 1.6 equiv.) in anhydrous diethyl ether (100 mL) cooled in an ice bath. After 2 hours, 2.0 mL water, 2.0 mL 15% w/v NaOH, and 5.0 mL water were added sequentially dropwise. After 15 min, the white suspension was diluted with DCM, gravity filtered through Celite®, and the solids were washed with DCM. The filtrate was concentrated (200 mbar, 25° C.) to afford 2-fluoro-2-methylpropanol as a colorless oil (2.09 g, 56.1%).

¹H NMR (300 MHz, CDCl₃) δ 1.34 (d, J=21.3 Hz, 6H), 1.95 (br t, 1H), 3.56 (dd, J=6.6, 20.7 Hz, 2H).

### Preparation of 2-fluoro-2-methylpropyl trifluoromethanesulfonate

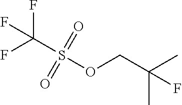

Trifluoromethanesulfonic anhydride (5.0 mL, 29.7 mmol, 1.3 equiv.) was added dropwise to a 0° C. solution of 2-fluoro-2-methylpropanol (2.090 g, 22.7 mmol, 1.0 equiv.) and 2,6 lutidine (3.40 mL, 29.4 mmol, 1.3 equiv.) in DCM (25 mL) over 30 minutes. After 2 hours, the red solution had turned light brown. TLC (20:80 EA:Hex, KMnO₄ stain) indicated that the starting material was not present. The reaction mixture was washed with 1M HCl solution (2×20 mL) and sat. NaHCO₃ solution (2×20 mL). The aqueous layers were each back extracted with DCM (20 mL). The combined organic layers were dried with Na₂SO₄, filtered and concentrated under reduced pressure (150 mbar, 25° C.) to afford 2-fluoro-2-methylpropyl trifluoromethanesulfonate as a red oil (4.39 g, 86.3%).

¹H NMR (300 MHz, CDCl₃) δ 1.46 (d, J=20.4 Hz, 6H), 4.41 (d, J=18.6 Hz, 2H). ¹⁹F NMR (282 MHz, CDCl₃) δ −147.1, −74.5.

### Preparation of (R)—N-(1-(1H-indol-3-yl)propan-2-yl)-2-fluoro-2-methylpropan-1-amine

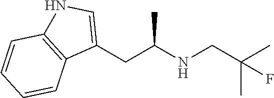 [SMILES: C[C@H](Cc1c[nH]c2ccccc12)NCC(C)(C)F]

The compound 2-fluoro-2-methylpropyl trifluoromethanesulfonate (9.587 g, 42.8 mmol, 1.1 equiv.) (solution in DCM, 16% DCM by wt %, 11.4384 g) was added to a solution of (2R)-1-(1H-indol-3-yl)propan-2-amine (6.680 g, 38.3 mmol, 1.0 equiv.), anhydrous 1,4-dioxanes (60.000 ml, 701.4 mmol, 18.3 equiv.), and freshly-distilled diisopropylethylamine (8.500 ml, 48.8 mmol, 1.3 equiv.). The dark brown solution was heated at 90° C. for 3 hours. After 3 h, LCMS indicated that a small amount of indolamine starting material was still present. TLC (10% MeOH/DCM) indicated triflate (Rf=0.54) had been used up. NMR of unused triflate SM (286-30) indicated the triflate had not decomposed overnight, so another 0.1 equiv (0.9883 g, 13% DCM wt %, 0.8563 g triflate SM) was added and the reaction was heated for 2 h at 90° C. LCMS indicated the reaction had completed and TLC (10% MeOH/DCM) showed one spot (Rf=0.24) (TLC with 50% EA/Hex, 1 streaked spot Rf<=0.12, another spot at Rf=0). EtOAc (50 mL) was added and the solution was washed with NaHCO₃ (2×50 mL) and the combined aqueous layer was washed with EtOAc (50 mL). The combined organic extracts were dried over Na₂SO₄ and concentrated under reduced pressure. The crude (brown oil, 14.8 g) was purified via flash silica chromatography (240 g cartridge, 0-100% EA/Hex). The desired product eluted as a long tailing peak. Pure fractions were concentrated to yield (R)—N-(1-(1H-indol-3-yl)propan-2-yl)-2-fluoro-2-methylpropan-1-amine (4.211 g, 17.0 mmol) as a dark yellow oil.

¹H NMR (300 MHz, CDCl₃) δ 1.10 (d, J=6.3 Hz, 3H), 1.34 (dd, J=3.0, 21.9 Hz, 6H), 2.68-2.95 (m, 4H), 3.02 (sextet, J=6.6 Hz, 1H), 7.05 (d, J=2.4 Hz, 1H), 7.26-7.11 (m, 2H), 7.36 (d, J=6.9 Hz, 1H), 7.62 (d, J=7.5 Hz, 1H), 8.18 (br s, 1H). ¹⁹F NMR (282 MHz, CDCl₃) δ −144.2. m/z: ES+ [M+H]+ 249.0.

### Preparation of Compound 1

4-((1-propylazetidin-3-yl)oxy)benzaldehyde (0.096 g, 0.4 mmol, 1.3 equiv.) was added to a solution of (R)—N-(1-(1H-indol-3-yl)propan-2-yl)-2-fluoro-2-methylpropan-1-amine (0.070 g, 0.3 mmol, 1.0 equiv.) in anhydrous toluene (1.50 mL) and glacial acetic acid (0.100 mL, 1.7 mmol, 6.2 equiv.). Molecular sieves were added and the solution was stirred under N₂ in the dark at 80° C. for 8 hours. The reaction solution was diluted in DCM, filtered, and washed with saturated Na₂CO₃ solution. The aqueous layer was extracted with DCM and the combined organic layers were dried over Na₂SO₄. The solution was filtered and concentrated. The residue was dissolved into acetonitrile (2 mL) and filtered through a syringe filter before purification via prep LC (40 to 90% ACN:H₂O over 18 min, followed by isocratic 90% ACN for 7 min). Pure fractions were concentrated and dried to afford (1R,3R)-2-(2-fluoro-2-methylpropyl)-3-methyl-1-(4-((1-propylazetidin-3-yl)oxy)phenyl)-2,3,4,9,-tetrahydro-1H-pyrido[3,4-b]indole as a white powder.

¹H NMR (300 MHz, CDCl₃) δ 0.90 (t, J=7.5 Hz, 3H), 1.09 (d, J=7.2 Hz, 3H), 1.26-1.50 (m, 8H), 2.45-2.77 (m, 6H), 3.01 (t, J=7.2 Hz, 2H), 3.34 (m, 1H), 3.77 (m, 2H), 4.60 (quin, J=5.7 Hz, 1H), 5.03 (s, 1H), 6.64 (d, J=8.1 Hz, 2H), 7.10-7.21 (m, 5H), 7.54 (d, J=7.5 Hz, 1H), 8.19 (br s, 1H). m/z: ES+ [M+H]⁺ 450.2.

### Example 2: Xenograft Analysis of Compound 1 in ST941 PDX Brain Metastases

The present example describes the effects of Compound 1 on tumors derived from the patient derived xenograft (PDX) model ST941 that have been implanted directly into mouse brains.

Among other things, this Example describes effects of Compound 1 on Estrogen Receptor (ER) positive tumors that have been implanted directly into mouse brains, using patient derived human breast cancer cells containing an activating mutation in the estrogen receptor, the Y537S ESR1 mutation.

Compared regimens included ovariectomy plus vehicle, fulvestrant, tamoxifen, Compound 1, and a combination of ribociclib and Compound 1, against vehicle in the ST941 intracranial breast cancer brain metastasis model.

### Protocol and Materials

### In Vitro Procedures

A tumor from a patient with subcutaneous ST941 tumor was used for intracranial inoculation of 64 athymic nude mice. The procedure was performed according to the following method: Subcutaneous ST941 PDX tumors grown in NMRI nude mice was harvested. The tumors were cut into small pieces and enzymatically digested to yield a suspension of single cells. The digestion was stopped, filtered through a 100 m filter, washed in PBS and resuspended in PBS. The viability of the tumor cells was checked by trypan blue staining and cells were suspended according the table below. The cells were kept on ice until inoculation.

| Inoculation | | | Tumor | | Inoculation |
|---|---|---|---|---|---|
| Site | Animal Strain | n | Model | Cells/animal | Medium |
| IC | Athymic Nude | 64 | ST941 | 200.000 in 10 μL | RPML 1640 |

### In Vivo Procedures

8 female NMRI nude mice (ordered age matched with a one week time frame, approximately 6 weeks of age) from Janvier Labs (France) were used in the model development.

### Tumor Implantation and Estrogen Administration

Tumors were prepared and implanted by the following method:

Animals were anaesthetized by hypnorm/midazolam (1 ml/100 g body weight) and placed in a stereotactic frame for fixation of the head. A longitudinal incision was made in the scalp exposing the calvarium. A hole was drilled in the skull 1.5 mm right of the sutura saggitalis and 1.0 mm posterior to the bregma using a micro-drill. 10 μl of the cell suspension (200.000 cells) was injected at a depth of 2-2.5 mm at a rate of 60 nl/sec using a 100 μl syringe with a 25-gauge needle placed in a micro infusion pump. The needle was left for 3 minutes before being withdrawn. Bupivacain (0.2 mg/100 g body weight) and Lidocain (1 mg/100 g body weight) was administrated in the incision site for local anesthetic and the skin was closed with a suture.

The animals were chipped for identification and returned to their cages where they were monitored until fully recovered from the anesthesia.

Mice were followed via MR imaging and were enrolled when tumor volume reached 2-5 mm³. Inclusion was performed either one or two days after MR imaging confirmed adequate tumor size. Mice were randomized such that all Groups had approximately equal tumor volume at enrollment. MR imaging was performed according to the following method:

The first MR imaging session was initiated 2 weeks after implantation. MR imaging was performed weekly or two times per week once tumor was established. Animals were enrolled when tumor volume reaches 2-5 mm³. Inclusion was the day after the actual MR imaging. Animals that met the enrollment criterion were stratified into 6 Groups according to Table 8-1. Animals were stratified so that all Groups have the same mean tumor volume at enrollment.

Estrogen supplement was provided via 17$-estradiol in drinking water from two days prior to intracranial inoculation. Estrogen supplement was withdrawn for each animal upon reaching sufficient tumor volume. Dosing for each mouse began two days after enrollment.

Table 8-1 summarizes treatments applied to different Groups:

TABLE 8-1

| Arm Code | Arm | Route | Dose | | Schedule | Animal Count |
|---|---|---|---|---|---|---|
| A | Vehicle | Oral Gavage | 0.0 | | QD for 49 | 8 |
| | | | | | days | |
| B | Ovariectomy + | Oral Gavage | 0.0 | | QD for 49 | 9 |
| | Vehicle | | | | days | |
| C | Fulvestrant | IM Injection | 5.0 | mg/mouse | QD for 49 | 8 |
| | | | | | days | |
| D | Tamoxifen | Oral Gavage | 60.0 | mg/kg | QD for 49 | 8 |
| | | | | | days | |
| E | Compound 1 + | Oral Gavage | 10.0 + 75.0 | | QD for 49 | 8 |
| | Ribociclib | | mg/kg | | days | |
| F | Compound 1 | Oral Gavage | 10.0 | mg/kg | QD for 49 | 9 |
| | | | | | days | |

QD = Once daily

The animals were treated according to Table 8-1. Therapy by oral gavage was given daily between 9 and 10 AM. The first dose was given two days after inclusion.

Compound 1 formulation was prepared by dissolving Compound 1 in DMSO to form a clear solution. This solution was transferred to a 0.5% CMC in millipore water such that the final concentration of DMSO was less than 5% v/v. During the addition of the DMSO solution, the Compound 1 precipitated to form a finely divided suspension in the vehicle. This suspension settled over time. Sonication and agitation were performed prior to administering the test article.

Ribociclib was formulated in an amount of 15 mg/mL. 100 mg ribociclib was suspended in 6.66 mL vehicle to reach a final concentration of 15 mg/mL.

A combination formulation of Compound 1 and ribociclib was prepared by mixing a 1:1 mL solution before administration.

### Ovariectomy

Animals in Group B were deprived of estrogen by an ovariectomy at the day of inclusion. Ovariectomy was performed according to the following method:

The animal was anesthetized (sevoflurane, 2-4% in ambient air supplemented with 100% O₂ at approximately 4:1 ratio) and placed in prone position on a heating pad. Carprofen (5 mg/kg) was administered subcutaneously before surgery and daily for 3 days post-surgery.

The area around the incision sites was disinfected using iodine. A 1 cm incision was made along the midline and the musculature was separated from the skin using curved scissors. A 1 cm lateral of the midline small incision was made through the musculature to reach the abdominal cavity. The white adipose tissue surrounding the ovary was removed using forceps. The proximal vessel and the uterine horn was ligated using a monofilament suture. The ovary was removed using a small scissor and the remaining tissue was placed back in the abdominal cavity.

The muscle layer wound and the skin layer wound were closed separately with resorbable sutures. The procedure was repeated on the opposite side.

### Therapy, Weight Monitoring and MR Imaging

Weight and visual assessment was monitored daily during therapy. The animals were scored three times per week according to the table below and more often if a weight loss of 10% was evident.

| Variable | Score |
|---|---|
| Body weight changes^{#} | |
| <15% | 0 |
| 15-20% | 2 |
| >20% | 3 |
| Body Condition Score (BCS) | |
| BCS > 3 | 0 |
| BCS ≤ 2 | 2 |
| Physical appearance | |
| Normal | 0 |
| Lack of grooming. | 1 |
| Ocular discharge | |
| Small bites or scratches. | 2 |
| Nasal discharge | |
| Serious bites or scratches. | 3 |
| Abnormal posture, limb, | |
| tremor etc. | |
| Unprovoked behavior | |
| Normal | 0 |
| Minor changes | 1 |
| Abnormal, reduced mobility, | 2 |
| decreased alertness, inactive | |

Tumor growth was monitored by MR on day 5, 10, 15, 20 and 25 relative to the day of inclusion.

The animals were euthanized by cervical dislocation when they met the humane endpoints.

### Tissue Preservation

Once the animals reached humane endpoint the following sampling was performed:
Blood (plasma sample)
   4 hours after last dosing whole blood was collected by cardiac puncture and transferred to an EDTA tube. Samples were centrifuged at 2000×g for 10 minutes at 4° C. Plasma was transferred to a 2 mL round bottom Eppendorf tube and stored at −80° C.
Brain with tumor tissue was resected.
   4 of the brains from each Group was preserved in 10% neutral buffered formalin for 48 hours (fixative ratio will be at least 1:20) and transferred to 70% ethanol. Samples were stored at 4° C. until shipment.
   4 of the brains from each Group were snap frozen and stored at −80° C. until shipment.

### Results

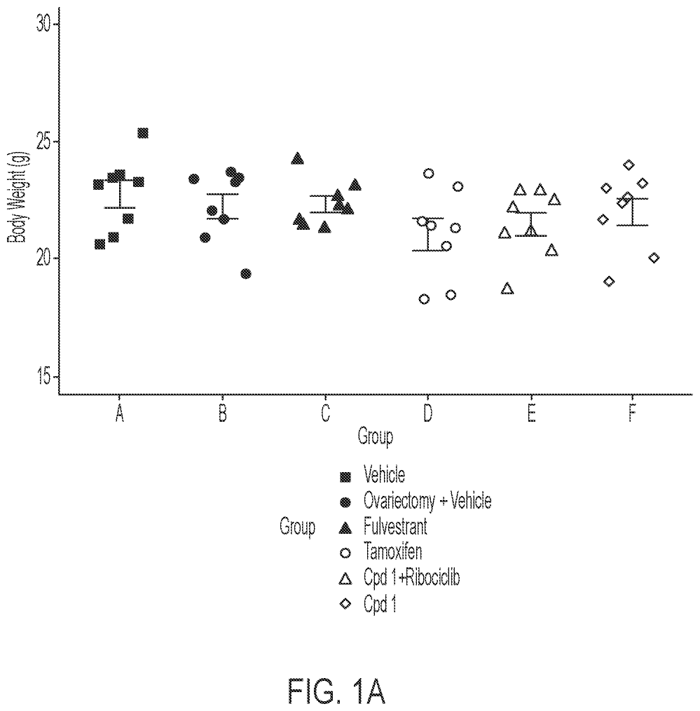

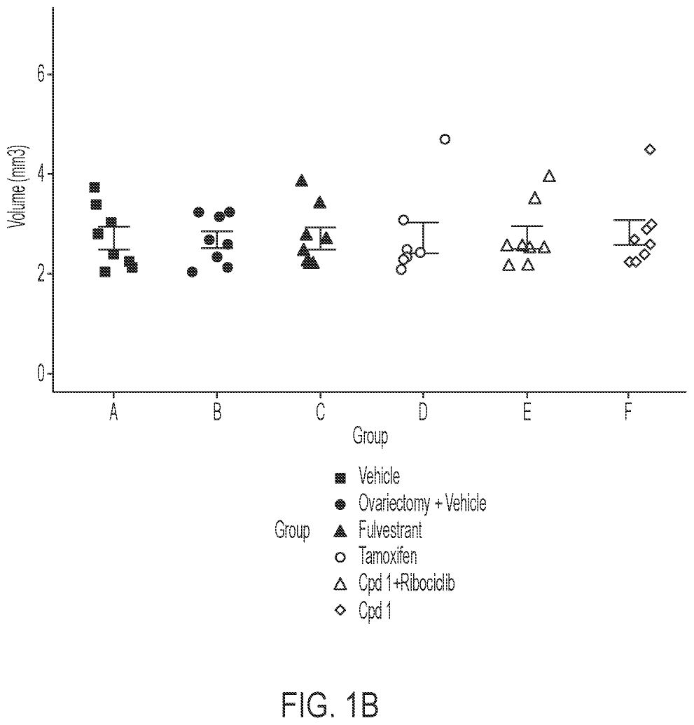

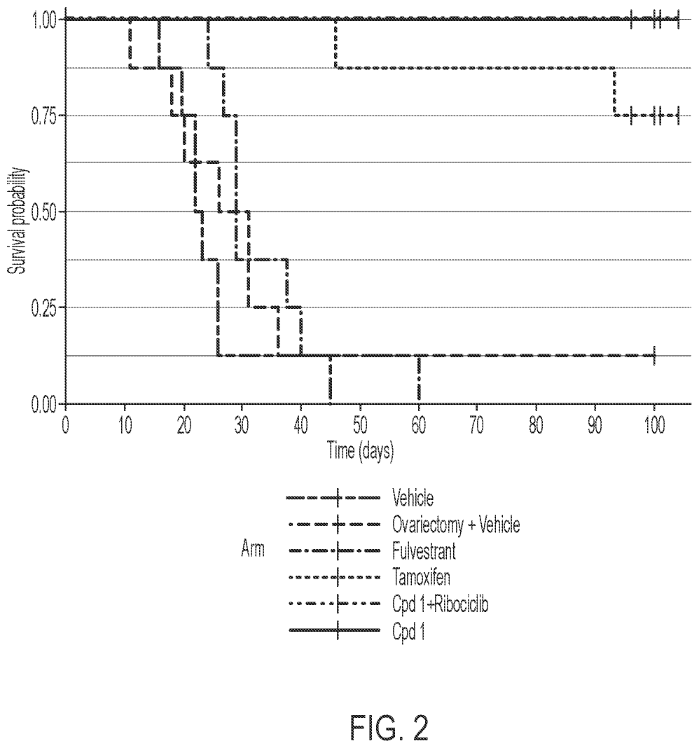

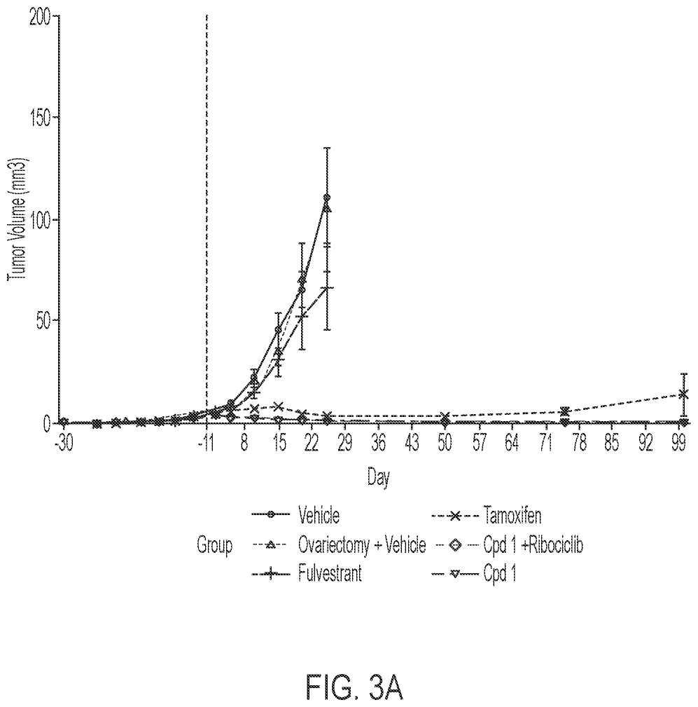

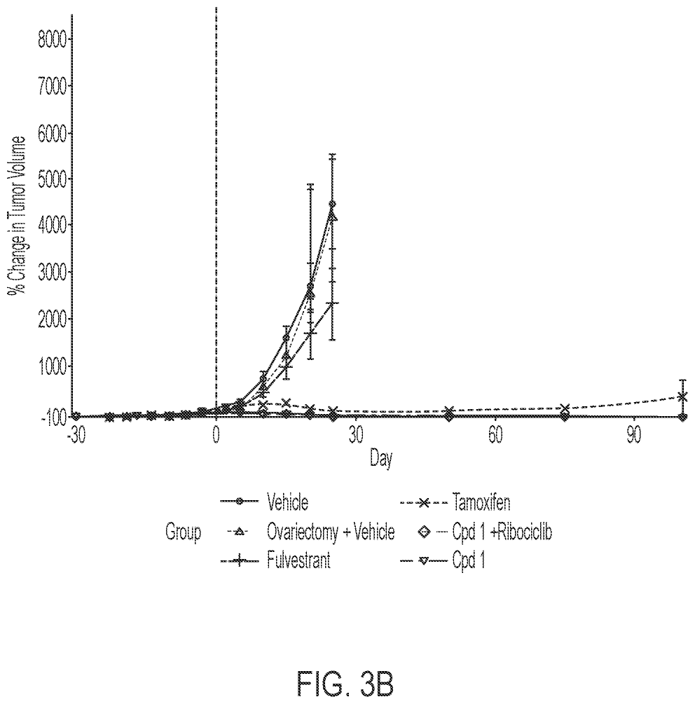

Results from the xenograft work described herein are provided in FIGS. 1A-8. These results illustrate that mice provided Compound 1, alone and in combination with ribociclib, exhibited improved survival rates as compared to other therapies, including fulvestrant. For example, almost all mice administered Compound 1 lived throughout the duration of the 100 day study, whereas at least half of the mice administered fulvestrant died in less than 30 days (see FIGS. 2, 5, and 6). The present disclosure, therefore, encompasses an insight that Compound 1, as a complete estrogen receptor antagonist, exhibits improved properties over fulvestrant. Tamoxifen was the only other therapy tested that slowed change in tumor volume, but not to the same extent as Compound 1 (see FIG. 3B). Animals treated with tamoxifen (Group D), however, after 100 days, started to see increases in tumor volume (see, e.g., FIGS. 3A, 3B, 5, 6), and the survival rate probability decreased after two members of the Group D died (see FIG. 2). The present disclosure also encompasses an insight that Compound 1 exhibits improved properties over tamoxifen in ability to treat brain metastases. Compound 1 is superior to tamoxifen for other reasons, including, but not limited to, Compound 1 being a complete estrogen receptor antagonist, whereas tamoxifen is a partial estrogen receptor agonist, as previously described.

FIG. 1A is a scatter plot measuring each animal's body weight at time 0. Throughout the Figures, “Cpd 1” refers to “Compound 1” as described herein.

FIG. 1B is a scatter plot measuring each animal's tumor volume at time 0.

FIG. 2 is a Kaplan-Meier plot showing the percentage of surviving mice in each Group over time. Tick marks indicate censored data reflecting that mice were enrolled on different days.

FIG. 3A is a scatter plot measuring mean tumor volume over time for the each Group.

FIG. 3B is a scatter plot measuring percent change in tumor volume over time for each Group.

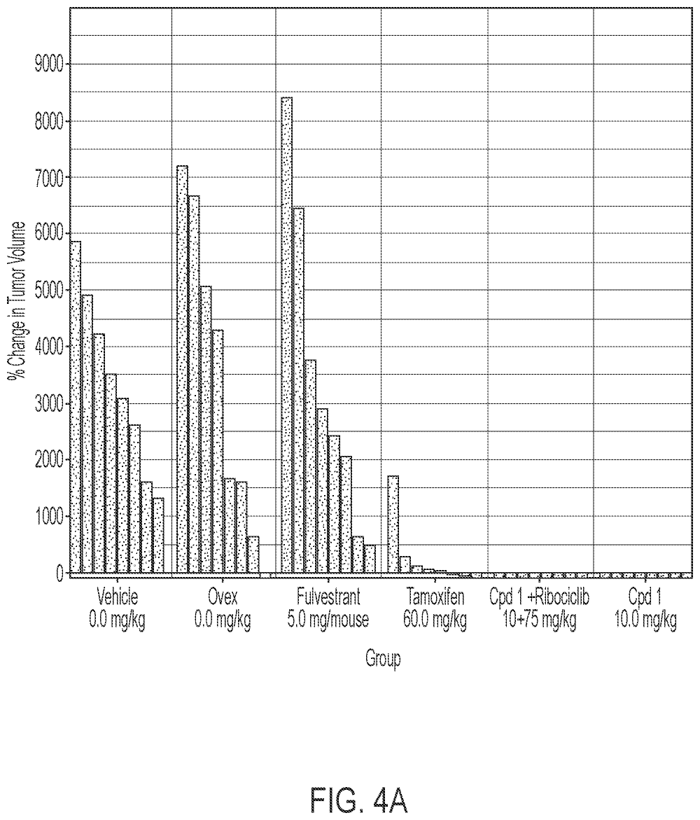

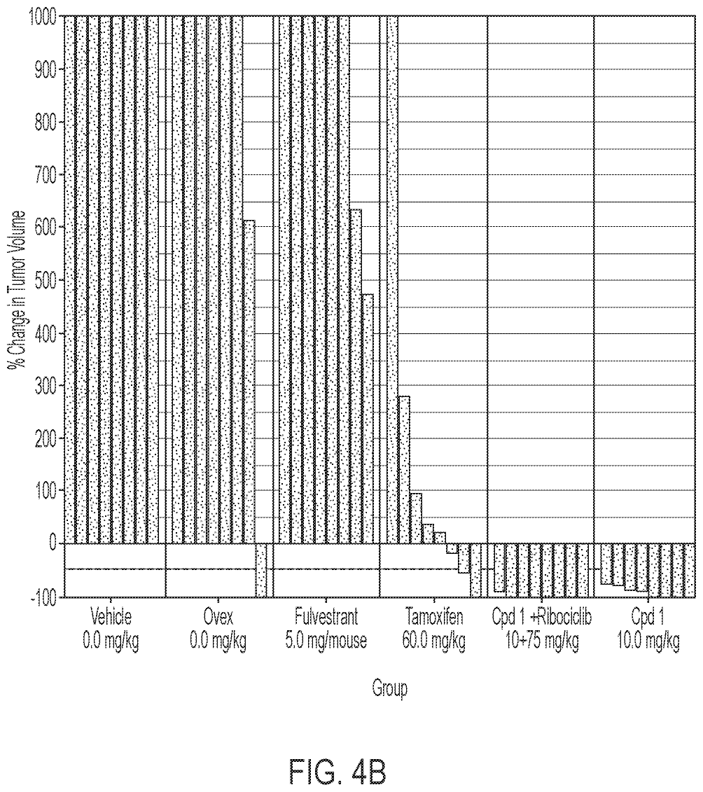

FIG. 4A is a waterfall plot measuring the percent change in tumor volume for each Group.

FIG. 4B is a zoomed in waterfall plot of FIG. 4A, illustrating the decrease in tumor volume size for certain Groups.

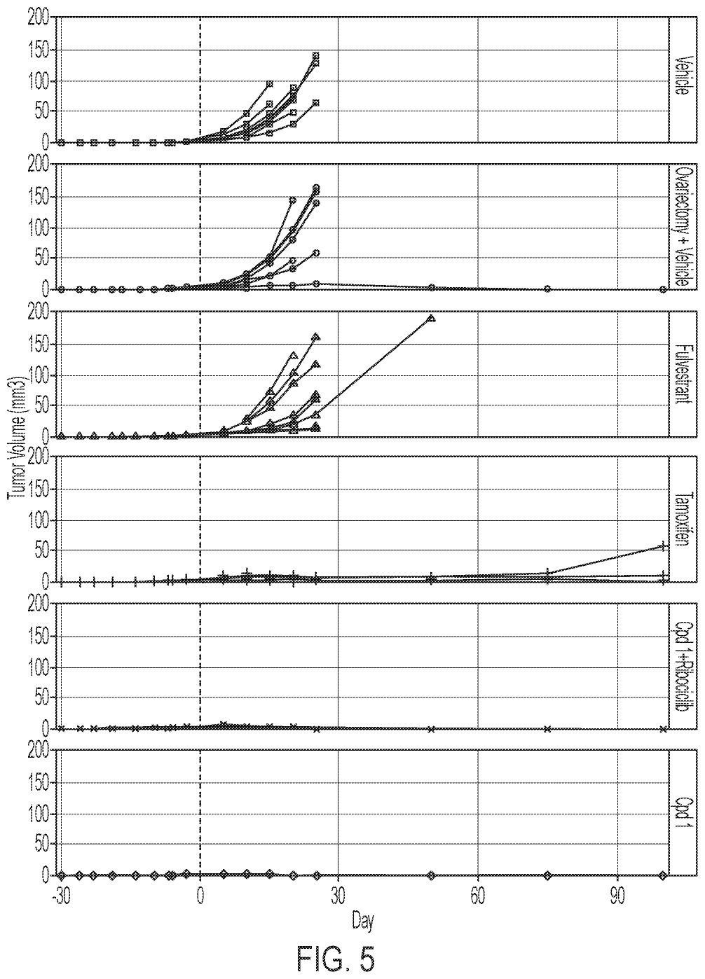

FIG. 5 is a scatter plot measuring mean tumor volume over time for individual animals of each Group.

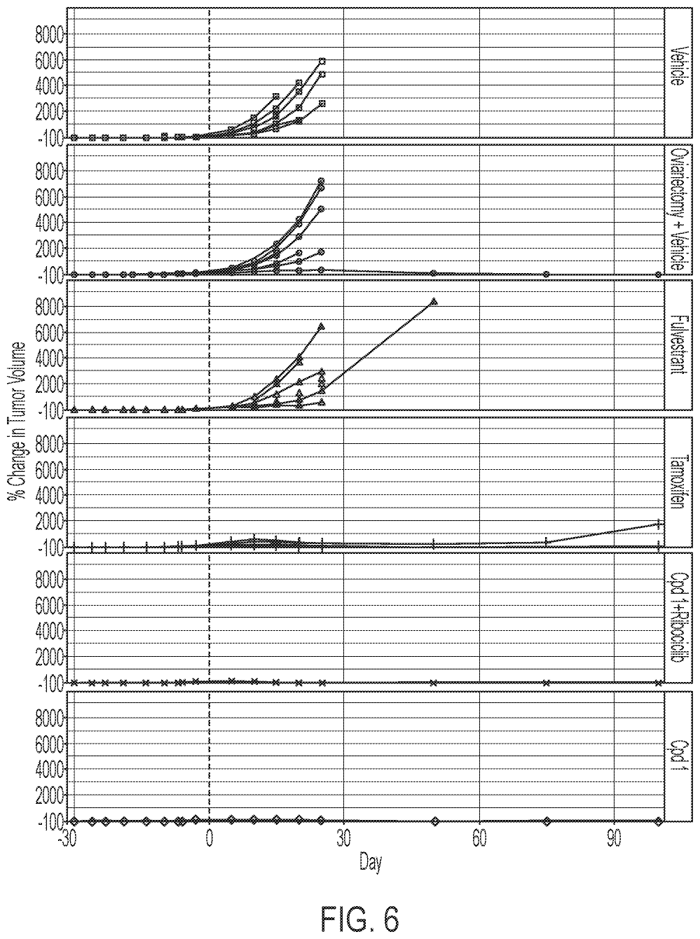

FIG. 6 is a scatter plot measuring percent change in tumor volume over time for individual animals of each Group.

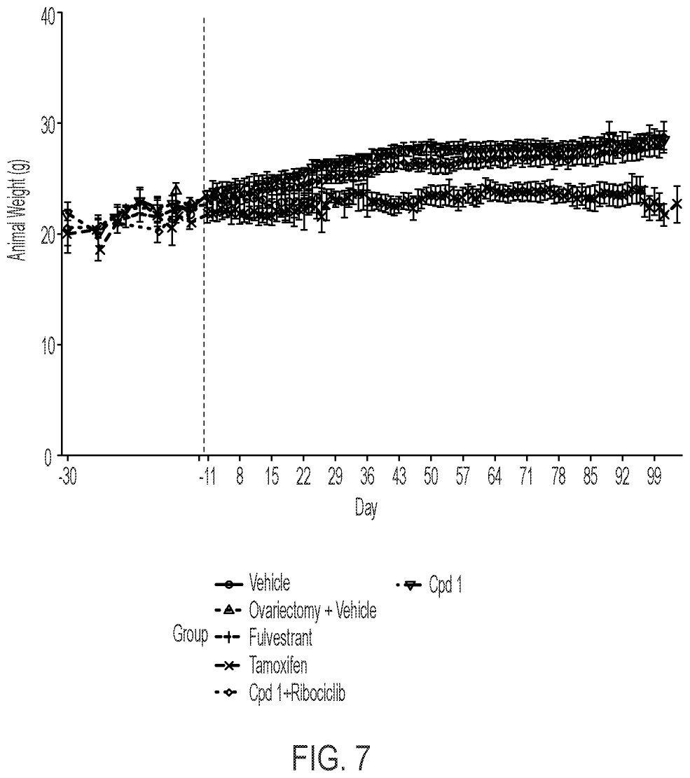

FIG. 7 is a scatter plot measuring animal weight over time for each Group.

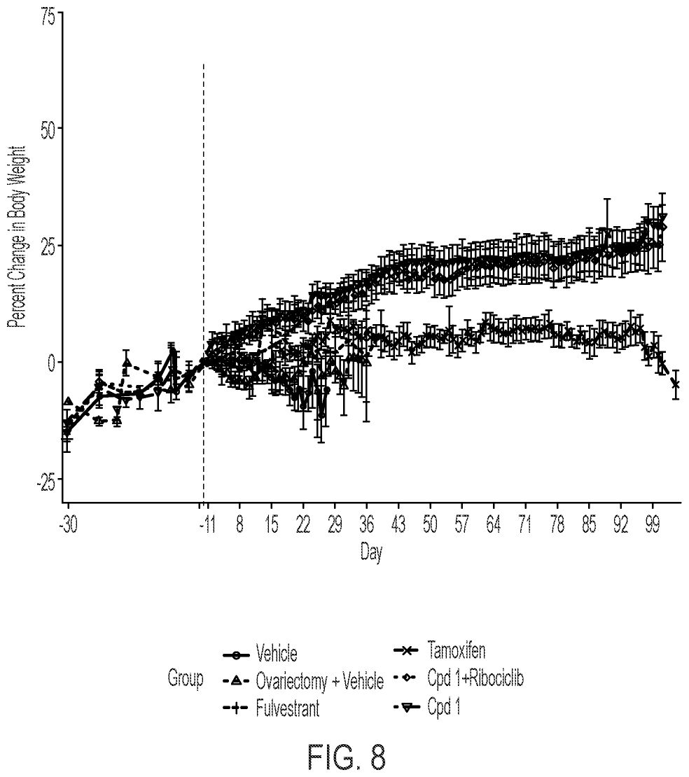

FIG. 8 is a scatter plot measuring percent change in animal weight over time for each Group.

### Example 3: Analysis of Compound 1 in Certain Cell Lines and Xenografts (BT-474, ZR-75-1, and ZR-75-30)

The present example describes the effects of Compound 1 on human breast cancer cell lines BT-474 (an ER+/HER2+ cell line), ZR-75-1 (an ER+/low HER2 cell line), and ZR-75-30 (an ER+/HER2+ cell line). Among other things, this Example describes the effects of Compound 1 on ER+ and HER2+ cell lines and tumors implanted into the mammary fat pad or flank of immunodeficient mice.

### Proliferation Assay

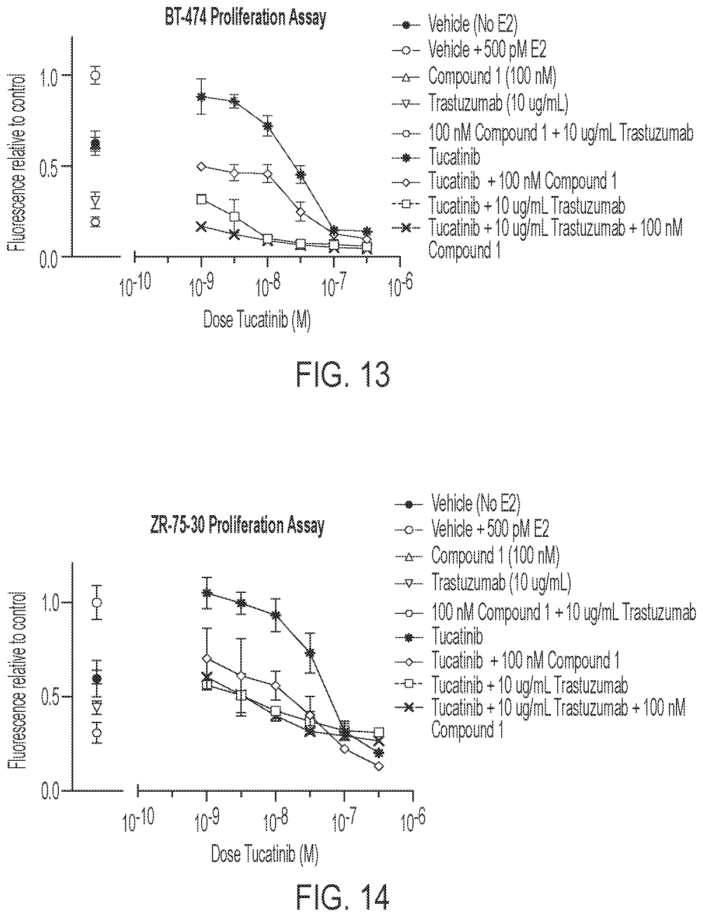

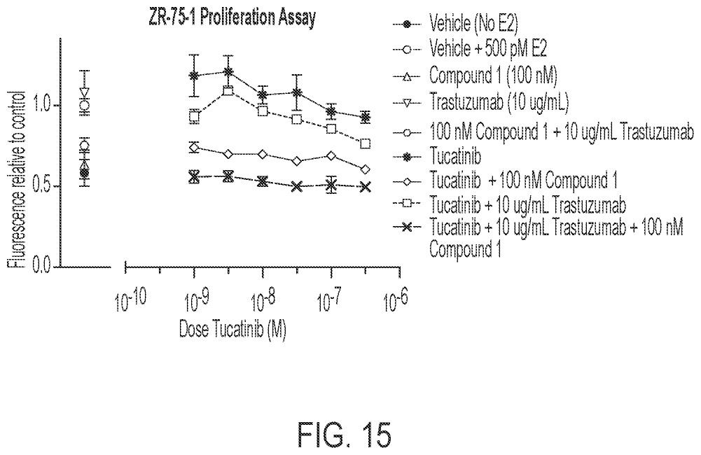

Proliferation of BT-474, ZR-75-30 or ZR-75-1 cells was measured using Cyquant, a fluorescent DNA-binding dye (Thermo Fisher Scientific). Cells were treated with compounds in triplicate in hormone-depleted medium for 7 days in the presence of 500 pM E2. Fluorescent activity was normalized to the activity of E2 alone and IC₅₀ was calculated using the least squares fit method. The results are provided in FIGS. 13-15. FIG. 13 is a plot analyzing fluorescence relative to control for varying doses of tucatinib in a BT-474 assay. FIG. 14 is a plot analyzing fluorescence relative to control for varying doses of tucatinib in a ZR-75-30 assay. FIG. 15 is a plot analyzing fluorescence relative to control for varying doses of tucatinib in a ZR-75-1 assay.

### Western Blot

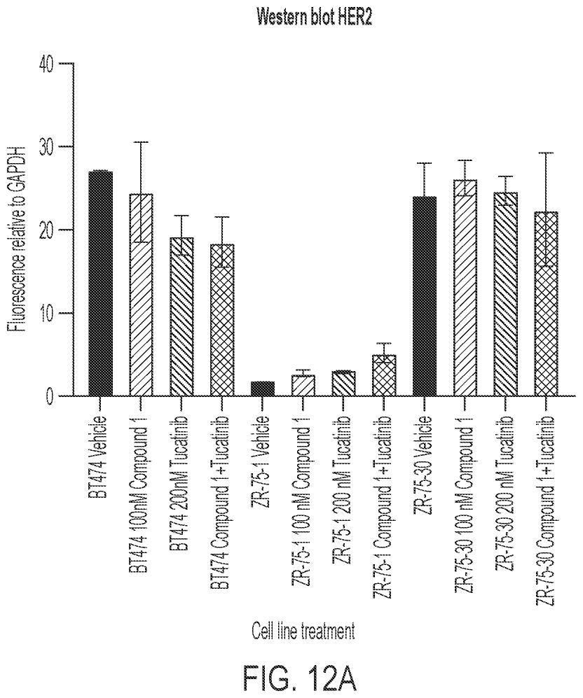

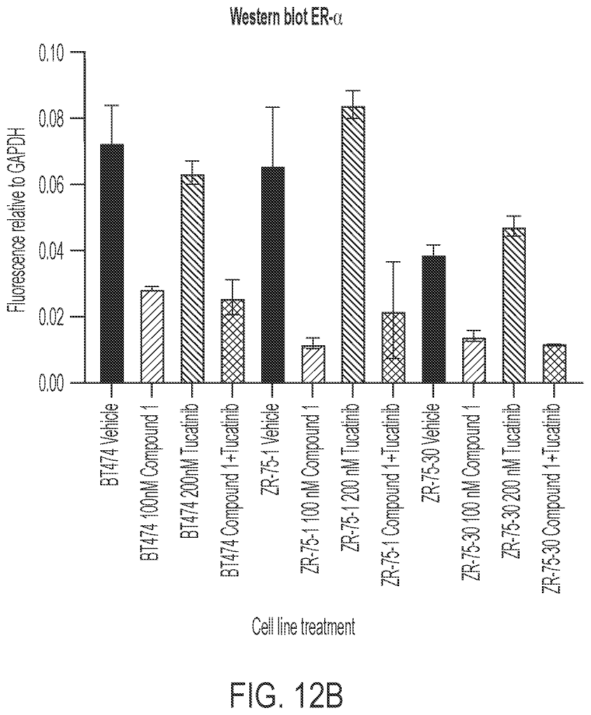

BT-474, ZR-75-30 or ZR-75-1 cells were treated with Compound 1 and tucatinib in duplicate for 4 hours in 5% charcoal dextran stripped serum medium and protein extracts immunoblotted with antibodies to HER2 (Invitrogen Ma5-14057) or ER-α (Thermo Fisher MA5-14501) and fluorescent secondary antibodies (LI-COR). Blots were imaged on a LI-COR Odyssey Fc imager and quantified using LI-COR Image Studio software. The results are provided in FIGS. 12A and 12B. FIG. 12A a chart tracking fluorescence relative to GAPDH in three cell lines, BT-474, ZR-75-1, and ZR-75-30 in a HER2 Western Blot. FIG. 12B is a chart tracking fluorescence relative to GAPDH in three cell lines, BT-474, ZR-75-1, and ZR-75-30 in a ERα Western Blot.

### BT-474 Xenograft Study

A xenograft study was conducted in accordance with the Institutional Animal Care and Use Committee (IACUC) guidelines. BT-474 cells were grown in culture and injected into the mammary fat pad of NSG immunodeficient mice. Beeswax estradiol pellets containing 0.44 mg estradiol were implanted to stimulate tumor growth. When tumors reached 100-205 mm³, mice were enrolled into treatment groups. One group received vehicle only (daily oral gavage 5% w/v solution of hydroxypropyl-beta-cyclodextrin (HPC) and 2.5% DMSO in water pH 4-5 and twice a week IP injection of 0.9% sodium chloride), while the remaining seven groups were treated with compounds either by daily oral gavage (Compound 1 and tucatinib) or by twice a week intraperitoneal injection (trastuzumab) with daily oral gavage vehicle. Compound 1 was dosed at 10 mg/kg, tucatinib at 50 mg/kg, and trastuzumab at 20 mg/kg. Compound 1 and tucatinib were co-formulated in 5% hydroxypropyl-beta-cyclodextrin (HPC). After 28 days of dosing study was terminated and mice euthanized. Mice were euthanized 24 hours post last dose and plasma, brain and tumor samples collected for analysis. Additional plasma samples were collected at 6 hours post last dose. Tumors and body weight were measured twice weekly. Tumor volumes were calculated with the formula Volume=(Width×Width×Length)/2.

Plasma, brain and tumor samples collected in BT-474 xenograft study were flash frozen for pharmacokinetic analysis. Tissue samples were homogenized with ice cold water and further diluted 2-fold with blank mouse plasma. An aliquot of plasma sample or plasma diluted tissue homogenate was extracted with methanol:acetonitrile (5:95, v:v) containing internal standard (verapamil) and mixed with 20% methanol for the injection to the LC/MS/MS. Calibration standards and quality control samples were prepared by spiking the test compound into blank mouse plasma and the resulting plasma was processed with the unknown samples in the same batch.

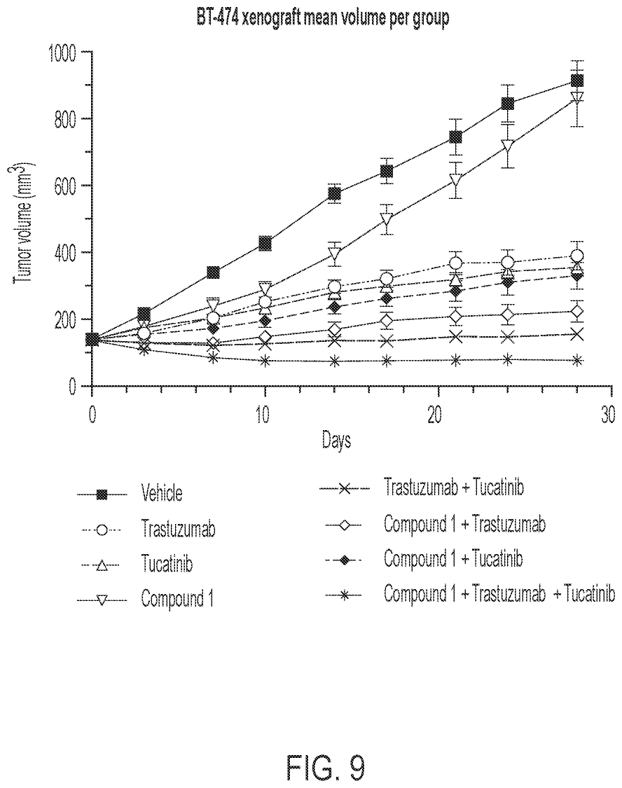

FIG. 9 is a scatter plot measuring change in BT-474 xenograft mean volume over time.

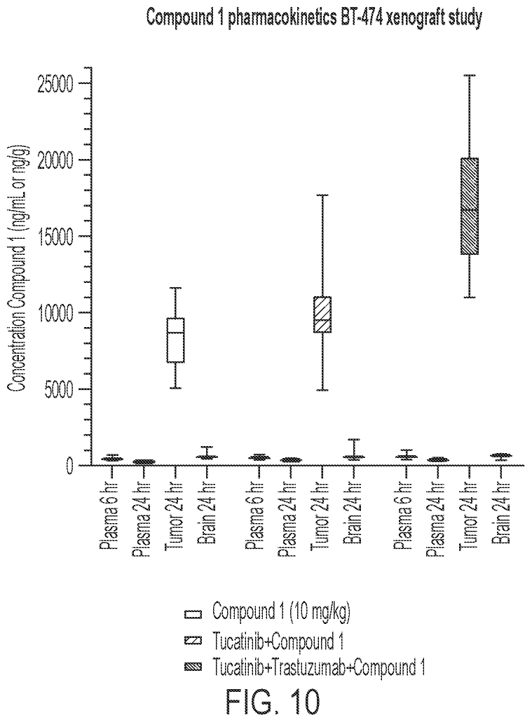

FIG. 10 is a graph measuring concentration of therapy in plasma and brain after 6 and 24 hours; demonstrating that Compound 1 exhibit brain penetrance.

### PDX Xenograft Study

When sufficient stock animals reached 1000-1500 mm³, tumors were harvested for re-implantation into pre-study animals. Immune-compromised athymic nude mice were implanted unilaterally on the left flank with tumor fragments harvested from stock animals. Each animal was implanted from a specific passage lot and documented. When tumors reached an average tumor volume of 150-300 mm³′ animals were matched by tumor volume into treatment or control groups.

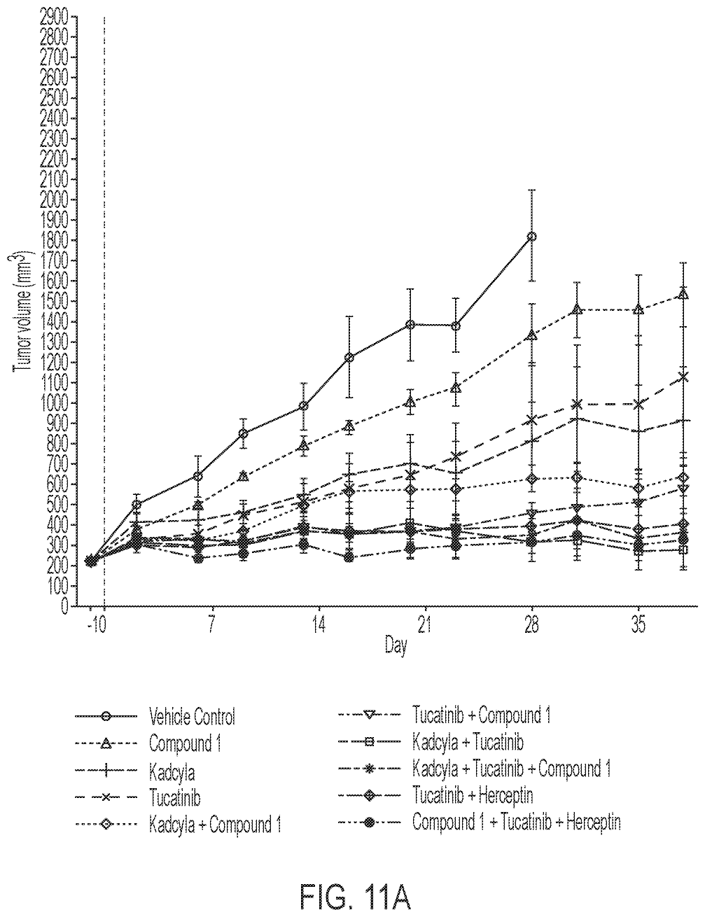

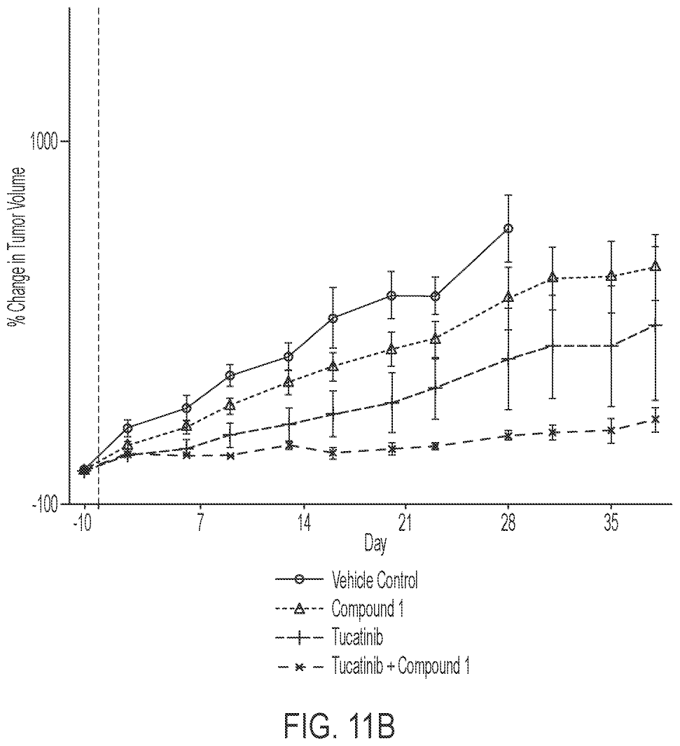

Tumor volumes were measured twice weekly. A final tumor volume was taken on the day study reached endpoint or if an animal was found moribund. Animals were weighed twice weekly. Any animal exhibiting >20% net weight loss for a period lasting 7 days or >30% net weight loss when compared to Day 0 was considered moribund and euthanized. Dosing was continued until Day 41 when remaining mice were euthanized. FIG. 11A is a scatter plot measuring change in xenograft tumor volume over time for provided therapies. Kadcyla is otherwise known as ado-trastuzumab emtansine. Herceptin is otherwise known as trastuzumab. FIG. 11B is a scatter plot measuring the percent change in xenograft tumor size over time for provided therapies.

### Example 4: Analysis of Compound 1 in ER+/HER2+ Cell Lines (BT-474, ZR-75-30, and MDA-MB-361) and Xenografts

The present example describes the effects of Compound 1 on human breast cancer cell lines BT-474, ZR-75-30, and MDA-MB-361. Among other things, this Example describes the effects of Compound 1 on ER+ and HER2+ cell lines and tumors implanted into the mammary fat pad or flank of immunodeficient mice.

### Proliferation Assay

Proliferation of BT-474, ZR-75-30 or MDA-MB-361 cells was measured using Cyquant, a fluorescent DNA-binding dye (Thermo Fisher Scientific). Cells were treated with compounds in triplicate in hormone-depleted medium for 7 days in the presence of 500 pM E2. Fluorescent activity was normalized to the activity of E2 alone.

### Western Blot

BT-474, ZR-75-30 or MDA-MB-361 cells were treated with 100 nM Compound 1 and/or 200 nM Tucatinib in duplicate for 4 hours in 5% charcoal dextran stripped serum medium and protein extracts immunoblotted with antibodies to HER2 (Invitrogen Ma5-14057), ER-α (Thermo Fisher MA5-14501), GAPDH (Cell Signaling 2118) and fluorescent secondary antibodies (LI-COR). Blots were imaged on a LI-COR Odyssey Fc imager and quantified using LI-COR Image Studio software. ER-α and HER2 signal was normalized to GAPDH signal for the corresponding sample and the results plotted using GraphPad Prism software.

### BT-474 Xenograft Study

BT-474 cells were grown in culture and injected into the mammary fat pad of NSG immunodeficient mice. Estradiol pellets were implanted to stimulate tumor growth. When tumors reached 100-200 mm³, mice were enrolled into treatment groups. One group received vehicle only (daily oral gavage 5% w/v solution of hydroxypropyl-beta-cyclodextrin (HPC) in water pH 4-5 and twice a week IP injection of PBS), while the remaining seven groups were treated with compounds either by daily oral gavage (Compound 1, Tucatinib and Capecitabine) or by twice a week intraperitoneal injection (Trastuzumab). Compound 1 was dosed at 10 mg/kg, Tucatinib at 50 mg/kg, Trastuzumab at 20 mg/kg, and Capecitabine at 175 mg/kg. Compound 1 and Tucatinib were either co-formulated in 5% hydroxypropyl-beta-cyclodextrin (HPC) or formulated separately in 5% HPC (capecitabine study). Dosing was terminated and samples collected after 28 days of treatment. Tumors and body weight were measured twice weekly.

For pharmacokinetic and immunohistochemistry analysis, mice were euthanized 24 hours post last dose and plasma, brain and tumor samples collected. Additional plasma samples were collected at 6 hours post last dose. Samples collected in BT-474 xenograft study were flash frozen for pharmacokinetic analysis. Tissue samples were homogenized with ice cold water and further diluted 2-fold with blank mouse plasma. An aliquot of plasma sample or plasma diluted tissue homogenate was extracted with methanol:acetonitrile (5:95, v:v) containing internal standard (verapamil) and mixed with 20% methanol for the injection to the LC/MS/MS. Calibration standards and quality control samples were prepared by spiking the test compound into blank mouse plasma and the resulting plasma was processed with the unknown samples in the same batch.

### CTG-3266 Xenograft Study

Pre-study animals were implanted unilaterally on the left flank with tumor fragments harvested from stock animals. Each animal is implanted from a specific passage lot and documented. When tumors reached an average tumor volume of 150-300 mm³, animals were matched by tumor volume into treatment or control groups.

Tumor volumes were measured twice weekly. A final tumor volume was taken on the day study reached endpoint or if an animal was found moribund. Animals were weighed twice weekly. Any animal exhibiting >20% net weight loss for a period lasting 7 days or >30% net weight loss when compared to Day 0 were considered moribund and euthanized. Dosing was continued until Day 41 when remaining mice were euthanized.

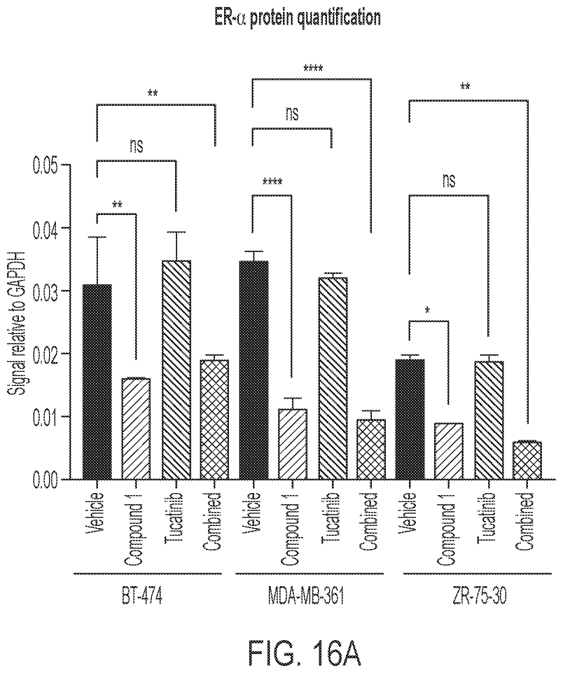

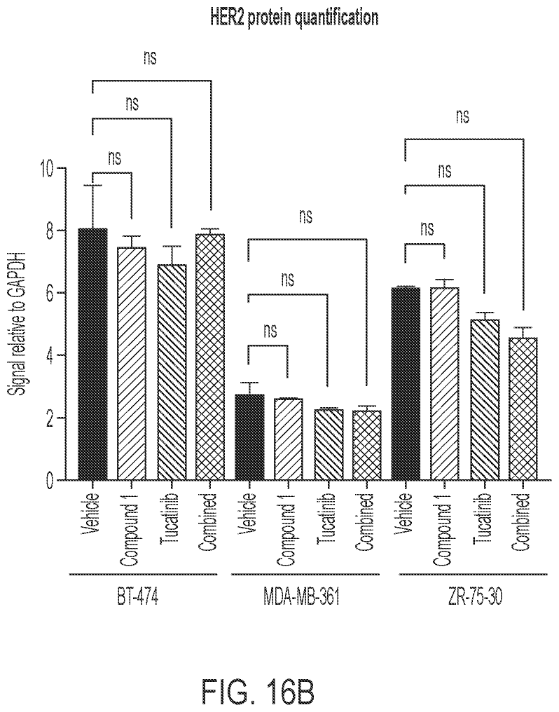

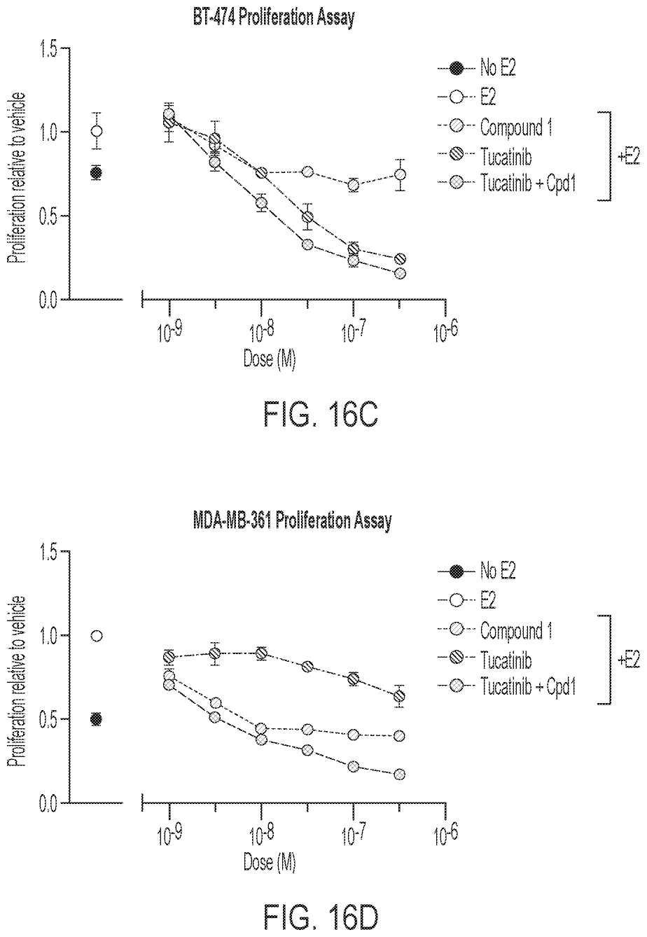

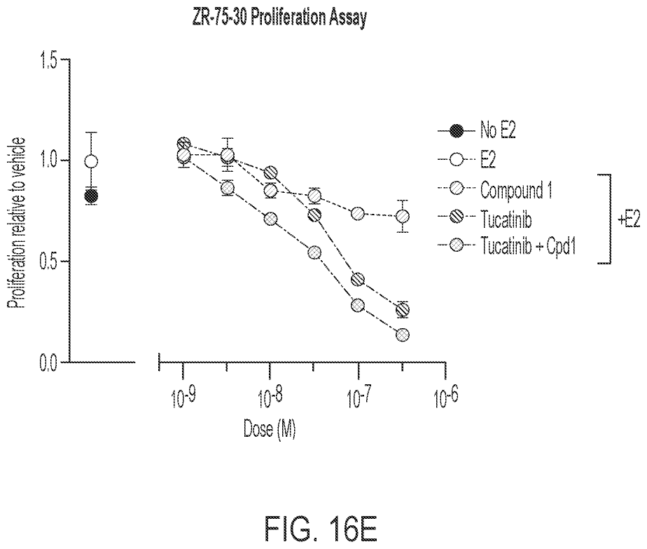

FIG. 16A is a bar graph illustrating ER-α protein quantification in BT-474, MDA-MB-361, and ZR-75-30 cell lines for certain compounds and combination.

FIG. 16B is a bar graph illustrating HER2 protein quantification in BT-474, MDA-MB-361, and ZR-75-30 cell lines for certain compounds and combination.

FIG. 16C is a scatter plot illustrating proliferation relative to vehicle in a BT-474 proliferation assay.

FIG. 16D is a scatter plot illustrating proliferation relative to vehicle in a MDA-MB-361 proliferation assay.

FIG. 16E is a scatter plot illustrating proliferation relative to vehicle in a ZR-75-30 proliferation assay.

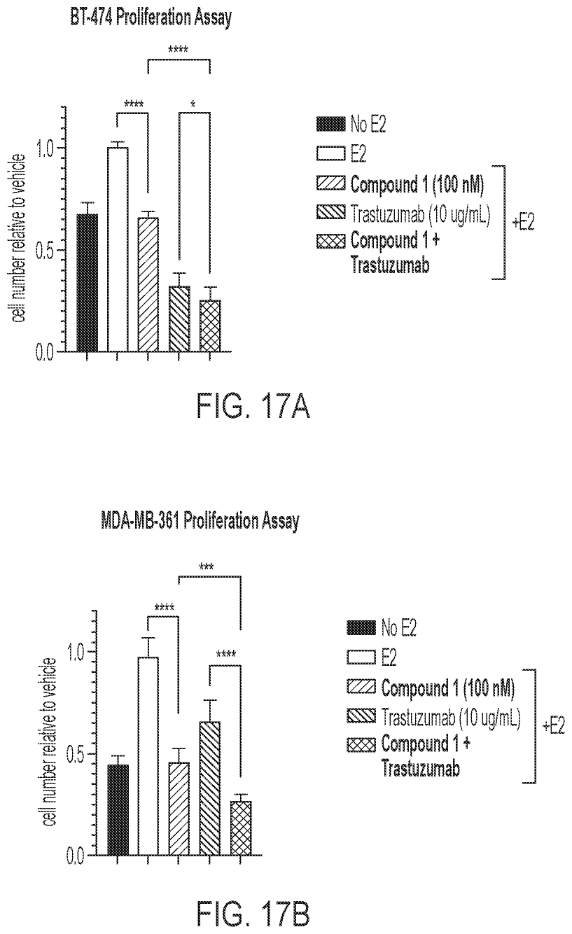

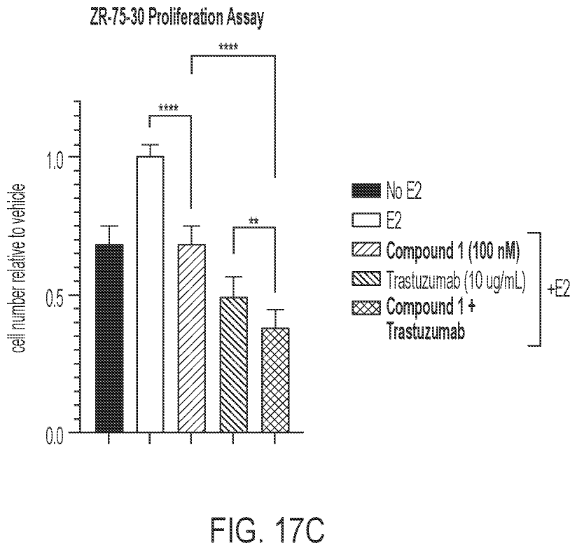

FIG. 17A is a bar graph illustrating cell proliferation in a BT-474 cell line for certain compounds and combinations.

FIG. 17B is a bar graph illustrating cell proliferation in a MDA-MB-361 cell line for certain compounds and combinations.

FIG. 17C is a bar graph illustrating cell proliferation in a ZR-75-30 cell line for certain compounds and combinations.

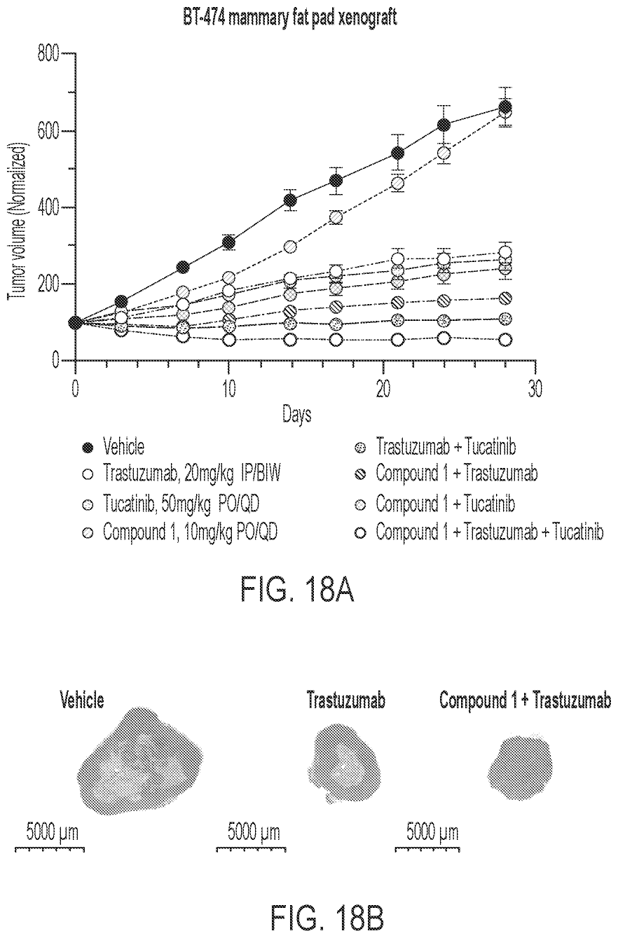

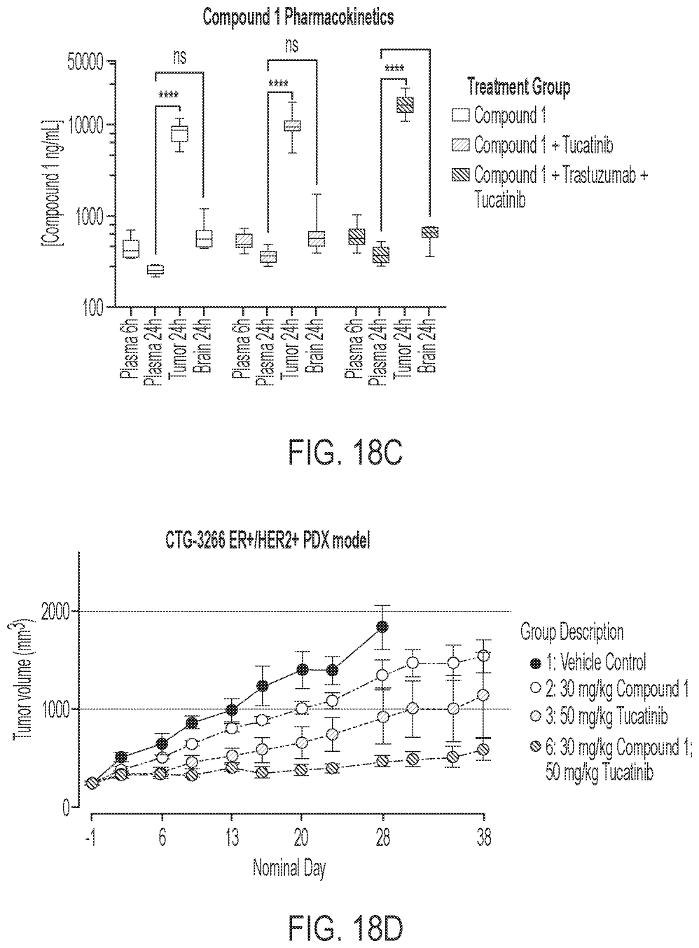

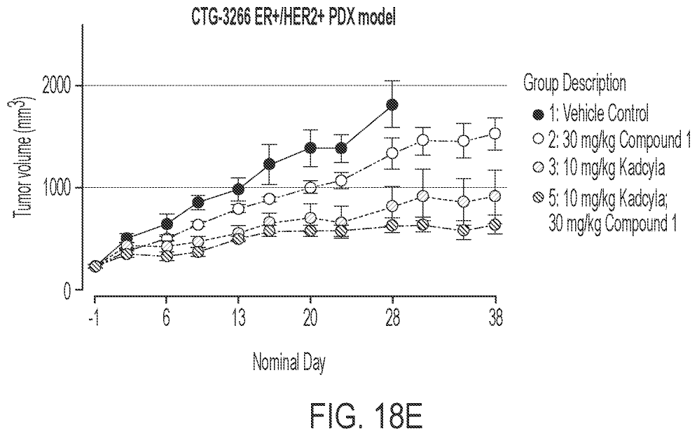

FIG. 18A is a scatter plot measuring tumor volume in a BT-474 mammary fat pad xenograft for certain compounds and combinations.

FIG. 18B is an image of various tumors treated with vehicle, trastuzumab, or Compound 1 and trastuzumab.

FIG. 18C is a graph illustrating concentration of Compound 1, tucatinib, and trastuzumab in the plasma, tumor, and brain.

FIG. 18D is a plot measuring tumor volume for ER+/HER2+ tumors treated with various compounds and combinations.

FIG. 18E is a plot measuring tumor volume for ER+/HER2+ tumors treated with various compounds and combinations.

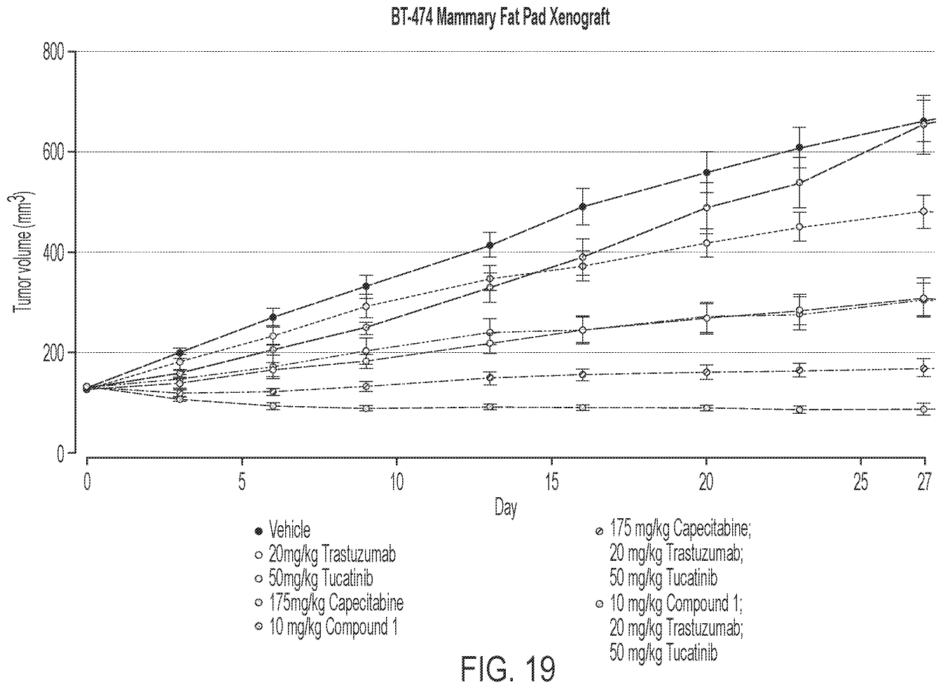

FIG. 19 is a plot measuring tumor volume in a BT-474 mammary fat pad xenograft for certain compounds and combinations.

### Example 5: Analysis of Compound 1 in ER+/HER2+ Cell Line (EFM192A) and Xenograft (CTG-2328)

The present example describes the effects of Compound 1 on human breast cancer cell lines EFM192A. Among other things, this Example describes the effects of Compound 1 on ER+ and HER2+ cell lines and tumors implanted into the mammary fat pad or flank of immunodeficient mice.

### Proliferation Assay

Proliferation of EFM192A cells was measured using Cyquant, a fluorescent DNA-binding dye (Thermo Fisher Scientific). Cells were treated with compounds in triplicate in hormone-depleted medium for 7 days in the presence of 500 pM E2. Fluorescent activity was normalized to the activity of E2 alone.

### CTG-2328 Xenograft Study

Pre-study animals were implanted unilaterally on the left flank with tumor fragments harvested from stock animals. Each animal was implanted from a specific passage lot and documented. When tumors reached an average tumor volume of 150-300 mm³, animals were matched by tumor volume into treatment or control groups. Tumor volumes were measured twice weekly. A final tumor volume was taken on the day study reached endpoint or if an animal was found moribund. Animals were weighed twice weekly. Any animal exhibiting >20% net weight loss for a period lasting 7 days or >30% net weight loss when compared to Day 0 was considered moribund and euthanized. Dosing was continued until Day 31.

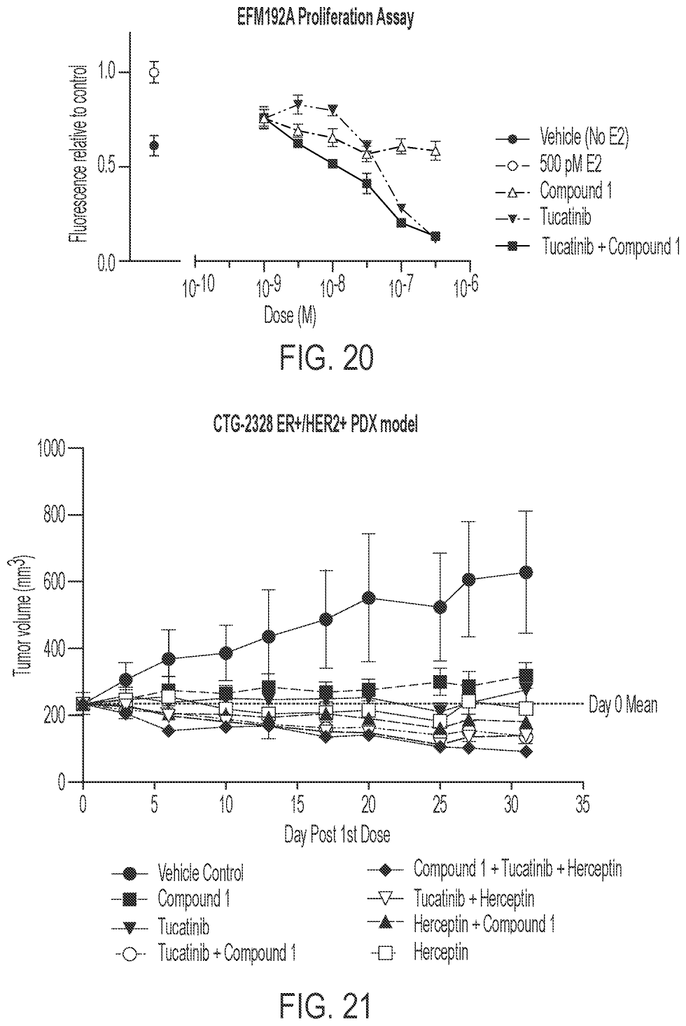

FIG. 20 is a scatter plot illustrating proliferation relative to vehicle in a EFM192A proliferation assay.

FIG. 21 is a scatter plot measuring tumor volume in a CTG-2328 xenograft for certain compounds and combinations. The dotted line indicates mean tumor volume at Day 0.

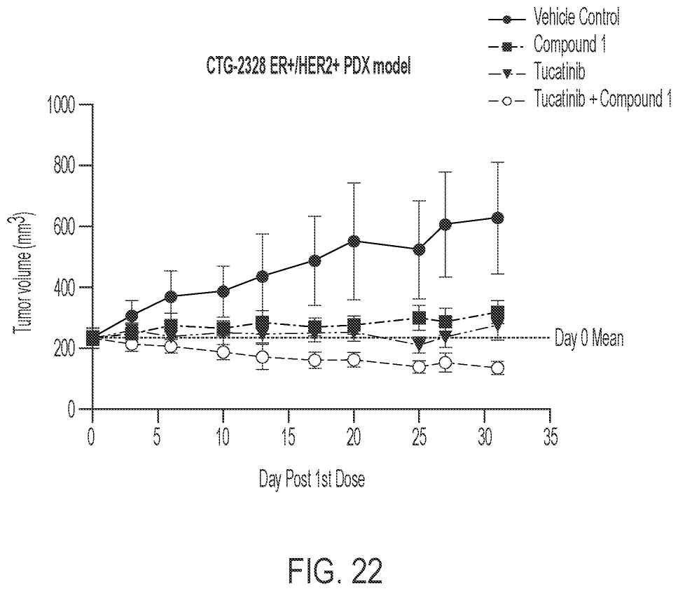

FIG. 22 is a scatter plot measuring tumor volume in a CTG-2328 xenograft for certain compounds and combinations. The dotted line indicates mean tumor volume at Day 0.

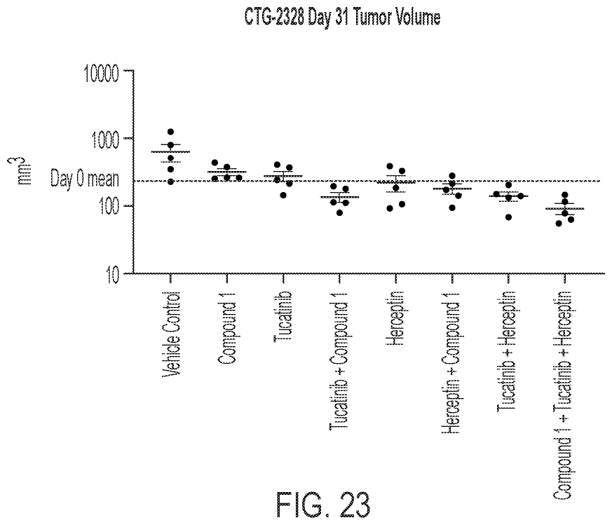

FIG. 23 is a plot measuring tumor volume on Day 31 in a CTG-2328 xenograft for certain compounds and combinations.

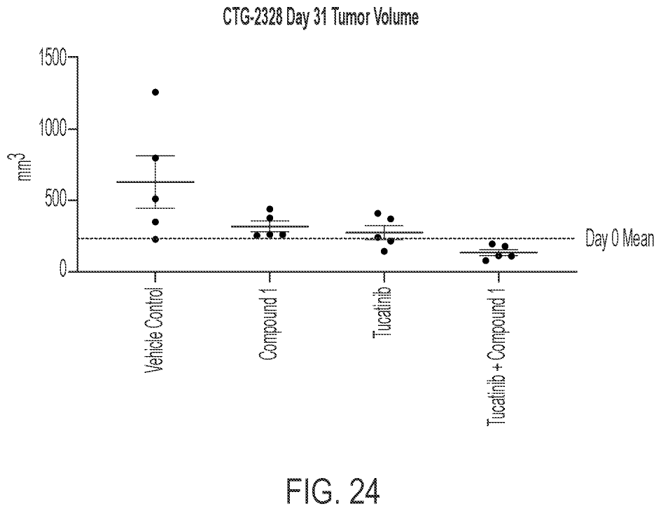

FIG. 24 is a plot measuring tumor volume on Day 31 in a CTG-2328 xenograft for certain compounds and combinations.

The foregoing has been a description of certain non-limiting embodiments of the subject matter described within. Accordingly, it is to be understood that the embodiments described in this specification are merely illustrative of the subject matter reported within. Reference to details of the illustrated embodiments is not intended to limit the scope of the claims, which themselves recite those features regarded as essential.

It is contemplated that systems and methods of the claimed subject matter encompass variations and adaptations developed using information from the embodiments described within. Adaptation, modification, or both, of the systems and methods described within may be performed by those of ordinary skill in the relevant art.

Throughout the description, where systems are described as having, including, or comprising specific components, or where methods are described as having, including, or comprising specific steps, it is contemplated that, additionally, there are systems encompassed by the present subject matter that consist essentially of, or consist of, the recited components, and that there are methods encompassed by the present subject matter that consist essentially of, or consist of, the recited processing steps.

It should be understood that the order of steps or order for performing certain action is immaterial so long as any embodiment of the subject matter described within remains operable. Moreover, two or more steps or actions may be conducted simultaneously.

1. A method of treating cancer in a subject determined to have an ER+ and HER2+ cancer, the method comprising:
administering to the subject Compound 1: 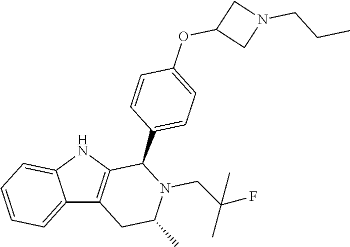 [SMILES: CCCN1CC(Oc2ccc([C@@H]3c4[nH]c5ccccc5c4C[C@@H](C)N3CC(C)(C)F)cc2)C1]
or a pharmaceutically acceptable salt thereof.

2. The method of claim 1, wherein the subject has been determined to have or is suspected of having metastases of the brain, bones, lungs or liver.

3. The method of claim 2, wherein the subject has been determined to have or is suspected of having brain metastases.

4. The method of any one of claims 1-3, wherein the ER+ and HER2+ cancer is breast cancer.

5. The method of claim 4, wherein the HER2 status of a primary tumor associated with the breast cancer and the metastases is the same.

6. The method of claim 5, wherein the HER2 status of a primary tumor associated with breast cancer and a brain metastasis is the same.

7. The method of claim 5, wherein the HER2 status of a primary tumor associated with breast cancer and a brain metastasis is the different.

8. The method of any one of claims 1-7, wherein the method further comprises administering one or more anti-cancer agents.

9. The method of claim 8, wherein the anti-cancer agent is a CDK 4/6 inhibitor, a PI3Kalpha inhibitor, an mTOR inhibitor, or an HER2 inhibitor.

10. The method of claim 9, wherein the anti-cancer agent is a CDK4/6 inhibitor.

11. The method of claim 10, wherein the CDK4/6 inhibitor is selected from palbociclib, ribociclib, abemaciclib, lerociclib, and trilaciclib.

12. The method of claim 11, wherein the CDK4/6 inhibitor is selected from ribociclib, palbociclib, and abemaciclib.

13. The method of claim 12, wherein the CDK4/6 inhibitor is palbociclib.

14. The method of claim 12, wherein the CDK4/6 inhibitor is ribociclib.

15. The method of claim 12, wherein the CDK4/6 inhibitor is abemaciclib.

16. The method of claim 9, wherein the anti-cancer agent is a PI3Kalpha inhibitor.

17. The method of claim 16, wherein the PI3Kalpha inhibitor is selected from alpelisib and taselisib.

18. The method of claim 9, wherein the anti-cancer agent is an mTOR inhibitor.

19. The method of claim 18, wherein the mTOR inhibitor is selected from sirolimus, temsirolimus, and everolimus.

20. The method of claim 9, wherein the anti-cancer agent is a HER2 inhibitor.

21. The method of claim 20, wherein the HER2 inhibitor is selected from tucatinib, trastuzumab, ado-trastuzumab emtansine, trastuzumab deruxtecan, pertuzumab, lapatinib, or neratinib.

22. The method of claim 21, wherein the HER2 inhibitor is tucatinib.

23. The method of claim 8, comprising administering two or more anti-cancer agents.

24. The method of claim 23, wherein one anti-cancer agent is a CDK4/6 inhibitor, and the other anti-cancer agent is a HER2 inhibitor.

25. The method of any one of claims 1-24, wherein the subject has previously been treated with an estrogen receptor inhibitor or an aromatase inhibitor.

26. The method of claim 25, wherein the estrogen receptor inhibitor is a selective estrogen receptor modulator or a selective estrogen receptor degrader.

27. The method of claim 26, wherein the estrogen receptor inhibitor is selected from tamoxifen, endoxifene, raloxifene, toremifene, lasofoxifene, ospemifene, and fulvestrant.

28. The method of claim 25, wherein the aromatase inhibitor is selected from letrozole, anastrozole, and exemestane.

29. The method of any one of claims 1-28, wherein Compound 1 is administered as a pharmaceutically acceptable salt.

30. The method of any one of claims 1-29, wherein Compound 1 is administered as a pharmaceutical composition comprising Compound 1, or a pharmaceutically acceptable salt thereof, and a pharmaceutically acceptable carrier.

31. The method of claim 30, wherein the pharmaceutical composition is a capsule.

32. The method of claim 31, wherein the pharmaceutical composition is a tablet.

33. The method of any one of claims 1-32, wherein Compound 1 is administered in a daily dose of from about 30 mg to about 360 mg.

34. The method of claim 33, wherein Compound 1 is administered in a daily dose of from about 60 mg to about 120 mg.

35. The method of any one of claims 1-34, wherein Compound 1 is administered orally.

36. The method of any one of claims 1-34, wherein Compound 1 is administered once daily.

37. In a method of treating cancer in a subject determined to have an ER+ and HER2+ cancer, the improvement comprising administering to the subject Compound 1: 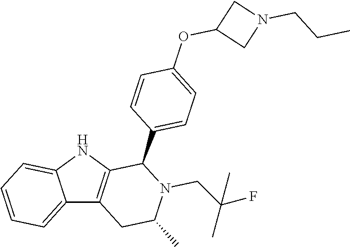 [SMILES: CCCN1CC(Oc2ccc([C@@H]3c4[nH]c5ccccc5c4C[C@@H](C)N3CC(C)(C)F)cc2)C1]
or a pharmaceutically acceptable salt thereof.

38. In a method of treating ER+ cancer comprising administering Compound 1: 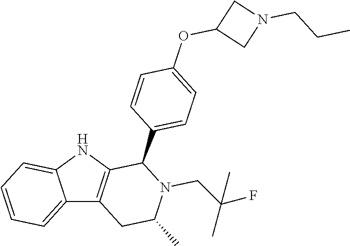 [SMILES: CCCN1CC(Oc2ccc([C@@H]3c4[nH]c5ccccc5c4C[C@@H](C)N3CC(C)(C)F)cc2)C1]
or a pharmaceutically acceptable salt thereof,
the improvement comprising administering Compound 1 or a pharmaceutically acceptable salt thereof to a subject determined to have an ER+ and HER2+ cancer.
